SEQUENCE LISTING (18 sequences)

SEQ ID NO: 1 - DNA, 649 bp, Oryza sativa
gccttcggaggccatgacactcgcctgctgaggtttgtcacgtacggcctgaagacgacgcttgtgttgtgcagtgcagtgttcggtctttcctggagcccacgagctcccatttgtgcgtgcgggcgcagccgccagagtcggcagaccggggaggtggcgcgcatgcacgcggtgaccactcctcctgcctccacgtacgtttgcctctggctttccctgctcattgctcatcgtcatcgtcatcggtgaggatgtctccggccggttcgggccgagcaagcagcaggttgcacggcccacacatgtaaggcctgtcacgtatgggctcggagaacggtttctttagcaggccgaatatccaggccctttttcactttttgccaaacactcaactgggccgcagcatcctcaactgcagctgcaagggtgaacatctgacctagctagatagatgctgccatagtgccatggcatattggcatggagtcgacgggatgaggtcaagatagtggacgtcgttggctcgttgcaagttgcaactggccaatcgtgctatagaaatcggtgctgattgggcggtatatccggttggatcgctcctaactccgaaattttgttaggtatgtcacgtcgtttccgcaactag

SEQ ID NO: 2 - DNA, 683 bp, Oryza sativa
ctacaccgtcgtctacgtcaaggacgtcgccaagtcggccgccttctactccgccgcgttcggctacaccgtccgccgcctcgaccaatcccacaagtaaaccatcctcggttaatattttttttccccgtcttaaatttttaactggctgcttgaaagtacttcctgatgcgtgtgacgtgtaatgaattataaggtgggcggagctggagagcgggacgacgacgatcgcgttcacgccgctgcaccagagggagacggacgcgctgacgggcgcggtgcagctgccggactcggccggcgagcgggggcccgtggagatctgcttcgactacgcggacgtcgacgcggcgtaccggcgggccgtggacagcggcgccgtgccggtgagcccgccggagcagaagagctggggccagaaggtcgggtacgtcagggacatcgacgggatcatcgtgcgcatgggcagccacgtccgcgcgtagcggccatgcgcctgctcggttgggggattttagccgtgtatgttcaataatgtgaactgttctccactgatctgttgatatatggaataaaactgtgatctcgttggttgtggtctgtactccaaagtccaaacgagaaaaaatggccatgtttgatgtatgatgtgttcaatcatgaatcacatgggtaaggatagg

SEQ ID NO: 3 - DNA, 11934 bp, Artificial Sequence
  The nucleotide sequence of vector DP0158
gaattctctagtcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaacgcggccgcttcagttgtggcccagcttggaggtcgactcgcgaggatctctgcagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgccaagctgctctaagctttggcggccgcattcgcaaaacacacctagactagatttgttttgctaacccaattgatattaattatatatgattaatatttatatgtatatggatttggttaatgaaatgcatctggttcatcaaagaattataaagacacgtgacattcatttaggataagaaatatggatgatctctttctcttttattcagataactagtaattacacataacacacaactttgatgcccacattatagtgattagcatgtcactatgtgtgcatccttttatttcatacattaattaagttggccaatccagaagatggacaagtctaggttaaccatgtggtacctacgcgttcgaatatccatgggccgctacaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttcgtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatggtggcgaggatctactcggctacactcacacgctcgctctcgcagttgcaggtgtaagtttctagctagggcactcacggggtacgtatttgtagccagccacgcacggtctgagctcgccatgtgccgccatgcatgcgggggcacgtcgccagcgtacgcggccatcgtcgctgacgaaggtagcgcattcaagtccggtcggtagaggtcagctgggtcgttcgccgatggtagttgccgcccggactcagtgggcggtaggcgaaggctagcaagcagacgactccattcatgcgcatcatccaaaggtgatgcaaagccttccaaacgcgattgtctcatgatgtttccgtctcttgttacgaggagtacaattttttcttatacacgaacgttactttatgtcacatttccatgccatgaacaccttggcttcaaataagtgagtgttttttttcacattctgtggcataaacagaatttctagagtggcatttgtgatacattgtgaaagctaagagtggtaaaagtaaaataaaattgttttgcttttgccgcggaatggaaattatttgtcaaaacctaagagtggcaaaactgaaatgtcaaaacctagagtgacataaacaaaatttacccatcactaaatgagcacaaaatatttcaccacaatggaggtatgtgaggtccgatgtactactagagctcatcggaaaagcatcctcttgatgagtaaacctcttgaagtactgtaccaccacattttatttatcctcatcggcttatttttaggccacggttattctcacgaagagacggttaacccttctcgtagactacacatcgagatccactagttctagagcggccagcttcgaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacg

SEQ ID NO: 4 - DNA, 867 bp, Oryza sativa
gaaacacacagcattgagactgtgagatcgagctatagtgacagccatggcaatcatcagtagcaaagcttgccgttgtttgctactcgtctcctttgccttgctcccactcagcatggccatggatccccttggcagctactgctccgggaacagcttggccggcagcagcaaggccgtggccagcatcaactccgtcctcaccgacctcgtcaccaagggctccaccggtgtcggcttcgccacgtccaccgccgggaaaggcaacaacgtcatctacggtctcgtgcaatgccgcggcgacgtctccaccagcgactgccaggcctgcctcgcctccgccgccaaccagatcctcaccagctgcaactaccaatccgactccagaatatggtatgactactgcttcatgcggttcgagaacgagaatttcttcgggcaggctgacacggacaacggggtgatcatggagaacgtgcaggcgatggacaacgccaaggcattccagaaggcggtggggaaggtgatgagcaaggcgacggcacaggtgtcacaggcgggaagcggcgggctgggaagggtgaaggaccaatacacgccattcatcaatatctacgggttcgcacagtgcacgcgggacctatcgccgctgacatgtgcacaatgcctatcaacggcggtgtccaggttcgaccaatattgcggcgcgcaacagggatgccggatactctacagtagctgcatggtgcgctacgaaatttaccccttctattttccgctcgccaccagtagcaccgccactaccgacatgacaaagtataccaagaccatcgtgcaccactaagaactacctacacatacatggtg

SEQ ID NO: 5 - DNA, 798 bp, Oryza sativa
atggcaatcatcagtagcaaagcttgccgttgtttgctactcgtctcctttgccttgctcccactcagcatggccatggatccccttggcagctactgctccgggaacagcttggccggcagcagcaaggccgtggccagcatcaactccgtcctcaccgacctcgtcaccaagggctccaccggtgtcggcttcgccacgtccaccgccgggaaaggcaacaacgtcatctacggtctcgtgcaatgccgcggcgacgtctccaccagcgactgccaggcctgcctcgcctccgccgccaaccagatcctcaccagctgcaactaccaatccgactccagaatatggtatgactactgcttcatgcggttcgagaacgagaatttcttcgggcaggctgacacggacaacggggtgatcatggagaacgtgcaggcgatggacaacgccaaggcattccagaaggcggtggggaaggtgatgagcaaggcgacggcacaggtgtcacaggcgggaagcggcgggctgggaagggtgaaggaccaatacacgccattcatcaatatctacgggttcgcacagtgcacgcgggacctatcgccgctgacatgtgcacaatgcctatcaacggcggtgtccaggttcgaccaatattgcggcgcgcaacagggatgccggatactctacagtagctgcatggtgcgctacgaaatttaccccttctattttccgctcgccaccagtagcaccgccactaccgacatgacaaagtataccaagaccatcgtgcaccactaa

SEQ ID NO: 6 - protein, 265 aa, Oryza sativa
MAIISSKACRCLLLVSFALLPLSMAMDPLGSYCSGNSLAGSSKAVASINSVLTDLVTKGSTGVGFATSTAGKGNNVIYGLVQCRGDVSTSDCQACLASAANQILTSCNYQSDSRIWYDYCFMRFENENFFGQADTDNGVIMENVQAMDNAKAFQKAVGKVMSKATAQVSQAGSGGLGRVKDQYTPFINIYGFAQCTRDLSPLTCAQCLSTAVSRFDQYCGAQQGCRILYSSCMVRYEIYPFYFPLATSSTATTDMTKYTKTIVHH

SEQ ID NO: 7 - DNA, 22 bp, Artificial Sequence
  Forward primer for cloning cDNA of OsCRK6 gene
gaaacacacagcattgagactg

SEQ ID NO: 8 - DNA, 23 bp, Artificial Sequence
  Reverse primer for cloning cDNA of OsCRK6 gene
caccatgtatgtgtaggtagttc

SEQ ID NO: 9 - DNA, 21 bp, Artificial Sequence
  Forward primer for real-time PCR analysis of OsCRK6 gene
gccactaccgacatgacaaag

SEQ ID NO: 10 - DNA, 21 bp, Artificial Sequence
  Reverse primer for real-time PCR analysis of OsCRK6 gene
gcatgcacatcaccatgtatg

SEQ ID NO: 11 - DNA, 1087 bp, Oryza sativa
ttttctgcctttcaggaaacttgctactttagttacttcttgtgtatagatttttgtctttttattattattttttttgcccccacatatattcatgtcacacgctgtattccaagtaaagaaagtatatagtaaaatttcatcacctgttatcagtctaatgaatctcttttacctttctgaaagttagatgatgattgttgttttgacaatccttaagcattttcatgatcatgttttacttctttcttttttttgataaaagagaggcttacaaaaatggatagtaccattacaaaaagacagtgctggtaaaacaaggcttagtttaaggctcctggtggtgccaaatcatgtgatcacataagctatgaatcatgttttactttagtgactatagtccactcttaacccatgctgtcctcagaaagcatttttcccacataagctaccagtcatcacaattgtcctgtcttcctcattactccgtggacacttgcaaccagttactgtgatcagaaactcagaataaggctgaagggttttgacttgtacgcgttaatgaacagaaaaatcacctttctgaaaaagactaggacgtaattgatgatgttaattgagtgataaccacagattccttctgatgatgagaggaaagttctctggggaaaaaaagtgaagatatggtgattcagagcaaaataaaacgggggacagatcttgaaaaggtttgggatggccctcgaagatatgctcatcagaagaaaaaggagaataattacttattgataaaccataaagttacctttcagatagctcagtaaagtaagcaccacccaagtaaataccgacagaatagtacggtagatttagtgaaaacagtcagacctcacaagtaaattagaaagttattactgcacatgtagaatgaatatggcatgaaaagaacaaatgcaaggagagaagacaaaaccaagttatagttcagaaatgcacgaatttcatattggtagcaaagtcttaaaagaggtcttcaggaaaagaaaaaggtaccaagcacctttttttacaagaaaaaataagtact

SEQ ID NO: 12 - DNA, 1746 bp, Oryza sativa
caacagcaaccactccgacgaacacctcgcctgcgcgatggtggcggcggcgctggacgcgatggcggggacgaggtgggggaggtggctggggctcgtgacggcggtgtgggtgcagtgcatctccggcaacaactacaccttctccaactactcccactccatcaagacgctcatgggcctcacccagctgcagctcaacggcctctccgtcgccaaggacgtcggcaaggcgttcggcctcctcgccggcctcgcctccgaccgcgtccccacctggcttctcctcgccgtcggctccctcgagggcctcctcggctacggcgcgcagtggctcgtcgtgtcccgggccgtcgcgccgctgccctactggcagatgtgcgtcttcctctgcctcggcgggaacagcacgacgtggatgaacaccgccgtgctcgtcacctgcatccgcaacttccgccggagcagggggccggtgtccgggttgctcaagggctatgtggggctcagcacggcgatcttcaccgacgtctgctccgcgctcttcgccgacgacccggcctcgttcctcgtcatgctcgccgtcgtgccggccgcggtgtgcgcggtcgccatggtgttcctccgcgagggggaggttggcggcggcggcgcggacgggcgggaggaggaggaggaggatgggtggtgcttcgccgcaatcaacacgctcgccgtcgccatcgcgctgtacctcctcgccgccgacctcaccggcgtcgggggaggcggcggggtcgtgtcggccgtcttcgtggccgtcctccttgtgctcctcgcgtcccccgccgcggtgccggcgcacgtggcgtggaagtcctggatgaagacgcggaagctcgcgaacgccgacgtcgaggaggcggaggagtgcgcgtccgccccgctcctcgtggcgaaggcgacggcggcggcggcggcggaggcgcgcggccccggcgagaagccggtgctcggggaggagcacacgatcgcgcaggcgctcatgtcgctggacttctggctcatgttcgcgtcgttcctgatgggcgtcggcacggggctcgccgtgatgaacaacctggggcagatgggcgtcgccatgggctactccgacgtctccctcttcgtctccatgaccagcatctggggattcttcggccgcatcgcctccgggaccatctccgagcacttcatcaagacaagagcaattccacgccccttgtggaatgcagcttcgcaaatcctgatggccgtgggctatgttgtgatggcagttggtatgccaggctccctcttcgtcggctccgttgtggttggcatctgctacggtgtccgcctggcggtcaccgtgccaacagcatctgaactgttcggtctcaagtactacggcctcatctacaacatcctcattctcaacttaccactcggctccttcctcttctctggccttcttgctggcctcctctacgacgcgcaggccaccaaggtacccggtggtggcaacacctgtgttggtgcgcactgctaccgcctcgtgttcgtggtcatggcgattgcctgcgtcgtcgggttcggtttggatgtcttgctgtgcttcaggaccaagagggtgtatgccaagatccatgagagcaagaggcagagcaggtcggcagttgtgcagagggtaagctagccaaaacctacagctatgagaaaaatggtacagattcgcgcaattgcatc

SEQ ID NO: 13 - DNA, 1659 bp, Oryza sativa
atggtggcggcggcgctggacgcgatggcggggacgaggtgggggaggtggctggggctcgtgacggcggtgtgggtgcagtgcatctccggcaacaactacaccttctccaactactcccactccatcaagacgctcatgggcctcacccagctgcagctcaacggcctctccgtcgccaaggacgtcggcaaggcgttcggcctcctcgccggcctcgcctccgaccgcgtccccacctggcttctcctcgccgtcggctccctcgagggcctcctcggctacggcgcgcagtggctcgtcgtgtcccgggccgtcgcgccgctgccctactggcagatgtgcgtcttcctctgcctcggcgggaacagcacgacgtggatgaacaccgccgtgctcgtcacctgcatccgcaacttccgccggagcagggggccggtgtccgggttgctcaagggctatgtggggctcagcacggcgatcttcaccgacgtctgctccgcgctcttcgccgacgacccggcctcgttcctcgtcatgctcgccgtcgtgccggccgcggtgtgcgcggtcgccatggtgttcctccgcgagggggaggttggcggcggcggcgcggacgggcgggaggaggaggaggaggatgggtggtgcttcgccgcaatcaacacgctcgccgtcgccatcgcgctgtacctcctcgccgccgacctcaccggcgtcgggggaggcggcggggtcgtgtcggccgtcttcgtggccgtcctccttgtgctcctcgcgtcccccgccgcggtgccggcgcacgtggcgtggaagtcctggatgaagacgcggaagctcgcgaacgccgacgtcgaggaggcggaggagtgcgcgtccgccccgctcctcgtggcgaaggcgacggcggcggcggcggcggaggcgcgcggccccggcgagaagccggtgctcggggaggagcacacgatcgcgcaggcgctcatgtcgctggacttctggctcatgttcgcgtcgttcctgatgggcgtcggcacggggctcgccgtgatgaacaacctggggcagatgggcgtcgccatgggctactccgacgtctccctcttcgtctccatgaccagcatctggggattcttcggccgcatcgcctccgggaccatctccgagcacttcatcaagacaagagcaattccacgccccttgtggaatgcagcttcgcaaatcctgatggccgtgggctatgttgtgatggcagttggtatgccaggctccctcttcgtcggctccgttgtggttggcatctgctacggtgtccgcctggcggtcaccgtgccaacagcatctgaactgttcggtctcaagtactacggcctcatctacaacatcctcattctcaacttaccactcggctccttcctcttctctggccttcttgctggcctcctctacgacgcgcaggccaccaaggtacccggtggtggcaacacctgtgttggtgcgcactgctaccgcctcgtgttcgtggtcatggcgattgcctgcgtcgtcgggttcggtttggatgtcttgctgtgcttcaggaccaagagggtgtatgccaagatccatgagagcaagaggcagagcaggtcggcagttgtgcagagggtaagctag

SEQ ID NO: 14 - protein, 552 aa, Oryza sativa
MVAAALDAMAGTRWGRWLGLVTAVWVQCISGNNYTFSNYSHSIKTLMGLTQLQLNGLSVAKDVGKAFGLLAGLASDRVPTWLLLAVGSLEGLLGYGAQWLVVSRAVAPLPYWQMCVFLCLGGNSTTWMNTAVLVTCIRNFRRSRGPVSGLLKGYVGLSTAIFTDVCSALFADDPASFLVMLAVVPAAVCAVAMVFLREGEVGGGGADGREEEEEDGWCFAAINTLAVAIALYLLAADLTGVGGGGGVVSAVFVAVLLVLLASPAAVPAHVAWKSWMKTRKLANADVEEAEECASAPLLVAKATAAAAAEARGPGEKPVLGEEHTIAQALMSLDFWLMFASFLMGVGTGLAVMNNLGQMGVAMGYSDVSLFVSMTSIWGFFGRIASGTISEHFIKTRAIPRPLWNAASQILMAVGYVVMAVGMPGSLFVGSVVVGICYGVRLAVTVPTASELFGLKYYGLIYNILILNLPLGSFLFSGLLAGLLYDAQATKVPGGGNTCVGAHCYRLVFVVMAIACVVGFGLDVLLCFRTKRVYAKIHESKRQSRSAVVQRVS

SEQ ID NO: 15 - DNA, 23 bp, Artificial Sequence
  Forward primer for cloning cDNA of OsMFS5 gene
caacagcaaccactccgacgaac

SEQ ID NO: 16 - DNA, 24 bp, Artificial Sequence
  Reverse primer for cloning cDNA of OsMFS5 gene
gatgcaattgcgcgaatctgtacc

SEQ ID NO: 17 - DNA, 20 bp, Artificial Sequence
  Forward primer for real-time PCR analysis of OsMFS5 gene
gttcggtttggatgtcttgc

SEQ ID NO: 18 - DNA, 20 bp, Artificial Sequence
  Reverse primer for real-time PCR analysis of OsMFS5 gene
ctctgcctcttgctctcatg

(19) United States
(12) Patent Application Publication
(10) Pub. No.: US 2019/0040412 A1
(43) Pub. Date: Feb. 7, 2019

(54) PLANTS HAVING ENHANCED TOLERANCE TO INSECT PESTS AND RELATED CONSTRUCTS AND METHODS INVOLVING INSECT TOLERANCE GENES

(71) Applicants: SINOBIOWAY BIO-AGRICULTURE GROUP CO. LTD., BEIJING (CN); PIONEER OVERSEAS CORPORATION, JOHNSTON, IA (US)

(72) Inventors: HUITING LI, BEIJING (CN); GUIHUA LU, BEIJING (CN); GUANFAN MAO, BEIJING (CN); GUOKUI WANG, BEIJING (CN); JINYU WANG, BEIJING (CN)

(73) Assignees: SINOBIOWAY BIO-AGRICULTURE GROUP CO. LTD., BEIJING (CN); PIONEER OVERSEAS CORPORATION, JOHNSTON, IA (US)

(21) Appl. No.: 16/070,836

(22) PCT Filed: Jan. 19, 2017
(86) PCT No.: PCT/CN2017/071679

§ 371 Date: Jul. 18, 2018

(30) Foreign Application Priority Data
Jan. 19, 2016 (CN) 201610040772.7

(51) Int. Cl. C12N 15/82; C07K 14/415; C12N 9/12; C12N 15/74

(52) U.S. Cl. CPC C12N 15/8286; C12N 15/743; C12N 9/1205; C07K 14/415

(57) ABSTRACT

The disclosure discloses isolated polynucleotides and polypeptides, and recombinant DNA constructs useful for conferring improved tolerance in plants to insect pests; compositions (such as plants or seeds) comprising these recombinant DNA constructs; and methods utilizing these recombinant DNA constructs. The recombinant DNA constructs comprise a polynucleotide operably linked to a promoter that is functional in a plant, wherein said polynucleotides encode insect tolerance polypeptides.

### FIELD

This disclosure relates to the field of plant breeding and genetics and, in particular, relates to recombinant DNA constructs useful for conferring tolerance to insect pests, and methods for control of insect infestation in plants.

### BACKGROUND

Numerous insect species are serious pests to common agricultural crops such as corn, soybean, pea, cotton, rice and similar food and fiber crops. Pests' infestation can cause a huge financial loss annually either in crop loss or in purchasing expensive pesticides to keep check on pests. During the last centuries, the primary method of controlling such pests has been through the application of synthetic chemical insecticidal compounds. However, the widespread use of chemical compounds poses many problems with regard to the environment because of the non-selectivity of the compounds and the development of insect resistance to the chemicals.

Advances in biotechnology in the last decades have presented new opportunities for pest control through genetic engineering. In particular, advances in plant genetics coupled with the identification of insect growth factors and naturally-occurring plant defensive compounds or agents offer the opportunity to create transgenic crop plants capable of producing such defensive agents and thereby protect the plants against insect attack.

Certain species of microorganisms of the genus Bacillus are known to possess pesticidal activity against a range of insect pests including Lepidoptera, Diptera, Coleoptera, Hemiptera and others. Bacillus thuringiensis (Bt) and Bacillus popilliae are among the most successful biocontrol agents discovered to date. Insect pathogenicity has also been attributed to strains of B. larvae, B. lentimorbus, B. sphaericus and B. cereus. Microbial insecticides, particularly those obtained from Bacillus strains, have played an important role in agriculture as alternatives to chemical pest control.

Transgenic plants that are resistant to specific insect pests have been produced using genes encoding Bacillus thuringiensis (Bt) endotoxins or plant protease inhibitors (PIs). For example, corn and cotton plants have been genetically engineered to produce pesticidal proteins isolated from strains of Bt. These genetically engineered crops are now widely used in agriculture and have provided the farmer with an environmentally friendly and commercially attractive alternative to traditional insect control methods. Generally speaking, the use of biopesticides presents a lower risk of pollution and environmental hazards and biopesticides provide greater target specificity than traditional broad spectrum chemical insecticides. In addition, biopesticides often cost less to produce and thus improve economic yield for a wide variety of crops.

While biopesticides have proven to be very successful commercially, these genetically engineered, insect-resistant crop plants provide resistance to only a narrow range of the economically important insect pests. In some cases, insects can develop resistance to different insecticidal compounds, which raises the need to identify alternative biological control agents for pest control. Accordingly, there remains a need for new pesticidal proteins with different ranges of insecticidal activity against insect pests, e.g., insecticidal proteins which are active against a variety of insects in the order Lepidoptera and the order Coleoptera including but not limited to insect pests that have developed resistance to existing insecticides.

### SUMMARY

In one aspect, the present disclosure includes an isolated polynucleotide enhancing insect tolerance of a plant through over-expression, comprising: (a) a polynucleotide with nucleotide sequence of at least 85% sequence identity to SEQ ID NO: 4 or 12; (b) a polynucleotide with nucleotide sequence of at least 85% sequence identity to SEQ ID NO: 5 or 13; (c) a polynucleotide encoding a polypeptide with amino acid sequence of at least 90% sequence identity to SEQ ID NO: 6 or 14; or (d) the full complement of the nucleotide sequence of (a), (b) or (c). The isolated polynucleotide comprises a nucleotide sequence of SEQ ID NO: 4, SEQ ID NO: 5, SEQ ID NO: 12 or SEQ ID NO: 13. The isolated polynucleotide encoded polypeptide comprising an amino acid sequence of SEQ ID NO: 6 or SEQ ID NO: 14. The said insect pest is a Lepidopteran, particularly Asian Corn Borer (Ostrinia furnacalis), Rice Stem Borer (Chilo suppressalis), or Oriental Armyworm (Mythimna separata).

In another aspect, the present disclosure includes a recombinant DNA construct comprising the isolated polynucleotide operably linked to at least one regulatory sequence, wherein the polynucleotide comprises (a) a polynucleotide with nucleotide sequence of at least 85% sequence identity to SEQ ID NO: 4, 5, 12 or 13; (b) a polynucleotide encoding a polypeptide with amino acid sequence of at least 90% sequence identity to SEQ ID NO: 6 or 14; or (c) the full complement of the nucleotide sequence of (a) or (b); the at least one regulatory sequence is a promoter functional in a plant.

In another aspect, the present disclosure includes a plant or seed comprising a recombinant DNA construct comprising the polynucleotide operably linked to at least one regulatory sequence, wherein the polynucleotide comprises (a) a polynucleotide with nucleotide sequence of at least 85% sequence identity to SEQ ID NO: 4, 5, 12 or 13; (b) a polynucleotide encoding a polypeptide with amino acid sequence of at least 90% sequence identity to SEQ ID NO: 6 or 14; or (c) the full complement of the nucleotide sequence of (a) or (b).

In another aspect, the present disclosure includes a plant comprising in its genome a recombinant DNA construct comprising a polynucleotide operably linked to at least one regulatory element, wherein the polynucleotide comprises (a) a polynucleotide with nucleotide sequence of at least 85% sequence identity to SEQ ID NO: 4, 5 12 or 13; (b) a polynucleotide encoding a polypeptide with amino acid sequence of at least 90% sequence identity to SEQ ID NO: 6 or 14; or (c) the full complement of the nucleotide sequence of (a) or (b); the said plant exhibits increased tolerance to an insect pest when compared to a control plant. The insect tolerance is created or enhanced against species of the orders selected from the group consisting of orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, etc., particularly Lepidoptera and Coleoptera. The said insect pest is Asian Corn Borer (Ostrinia furnacalis), Rice Stem Borer (Chilo suppressalis), or Oriental Armyworm (Mythimna separata). The present disclosure includes any of the plants of the disclosure, wherein the plant is selected from the group consisting of rice, maize, soybean, sunflower, sorghum, canola, wheat, alfalfa, cotton, barley, millet, sugar cane and switchgrass.

In another aspect, methods are provided for increasing tolerance in a plant to an insect pest, comprising: (a) introducing into a regenerable plant cell a recombinant DNA construct comprising a polynucleotide operably linked to at least one regulatory sequence, wherein the polynucleotide encodes a polypeptide having an amino acid sequence of at least 80% sequence identity compared to SEQ ID NO: 6 or 14; (b) regenerating a transgenic plant from the regenerable plant cell after step (a), wherein the transgenic plant comprises in its genome the recombinant DNA construct; and (c) obtaining a progeny plant derived from the transgenic plant of step (b), wherein the said progeny plant comprises in its genome the recombinant DNA construct and exhibits increased tolerance to an insect pest when compared to a control plant not comprising the recombinant DNA construct. In another aspect the insect pest is a Lepidopteran, particularly Asian Corn Borer (Ostrinia furnacalis), Rice Stem Borer (Chilo suppressalis), or Oriental Armyworm (Mythimna separata).

In another aspect, methods are provided for evaluating tolerance in a plant to an insect pest, comprising: (a) introducing into a regenerable plant cell a recombinant DNA construct comprising a polynucleotide operably linked to at least one regulatory sequence, wherein the polynucleotide encodes a polypeptide having an amino acid sequence of at least 80% sequence identity when compared to SEQ ID NO: 6 or 14; (b) regenerating a transgenic plant from the regenerable plant cell after step (a), wherein the transgenic plant comprises in its genome the recombinant DNA construct; (c) obtaining a progeny plant derived from the transgenic plant, wherein the progeny plant comprises in its genome the recombinant DNA construct; and (d) evaluating the progeny plant for tolerance to an insect pest compared to a control plant not comprising the recombinant DNA construct. In another aspect the insect pest is a Lepidopteran, particularly Asian Corn Borer (Ostrinia furnacalis), Rice Stem Borer (Chilo suppressalis) or Oriental Armyworm (Mythimna separata).

In another aspect, the present disclosure concerns a recombinant DNA construct comprising any of the isolated polynucleotides of the present disclosure operably linked to at least one regulatory sequence, and a cell, a plant, and a seed comprising the recombinant DNA construct. The cell may be eukaryotic, e.g., a yeast, insect or plant cell, or prokaryotic, e.g., a bacterium.

### BRIEF DESCRIPTION OF THE DRAWINGS AND SEQUENCE LISTINGS

The disclosure can be more fully understood from the following detailed description and the accompanying drawings and Sequence Listing which form a part of this application.

FIG. 1 shows the relative expression levels of OsCRK6 gene in leaves of different transgenic rice lines by real-time PCR analyses. The base expression level in ZH11-TC is set at 1.00, the numbers on the top of the columns are fold-changes compared to ZH11-TC rice. ZH11-TC is tissue cultured Zhonghua 11 and DP0158 is Zhonghua 11 rice transformed with empty vector.

FIG. 2 shows the relative expression levels of OsMFS6 gene in leaves of different transgenic rice lines by real-time PCR analyses. The base expression level in ZH11-TC is set at 1.00, the numbers on the top of the columns are fold-changes compared to ZH11-TC rice. ZH11-TC is tissue cultured Zhonghua 11 and DP0158 is Zhonghua 11 rice transformed with empty vector.

Table 1. SEQ ID NOs for nucleotide and amino acid sequences provided in the sequence listing

Table 2. Scoring Scales for Asian corn borer and Oriental armyworm assays

Table 3. Asian corn borer assay of AH43610 seedlings under laboratory screening condition

Table 4. Asian corn borer assay of AH29691 seedlings under laboratory screening condition

Table 5. Oriental armyworm assay of AH43610 and AH29691 seedlings under laboratory screening condition

Table 6. Rice stem borer assay of AH43610 and AH29691 seedlings under laboratory screening condition

Table 7. Primers for cloning insect tolerance genes

Table 8. PCR reaction mixture

Table 9. PCR cycle conditions for cloning insect tolerance gene

Table 10. Asian corn borer assay of OsCRK6 transgenic rice under laboratory screening condition at line level (1^{st} experiment)

Table 11. Asian corn borer assay of OsCRK6 transgenic rice under laboratory screen condition at line level (2^{nd} experiment)

Table 12. Armyworm assay of OsCRK6 transgenic rice under laboratory screen condition at construct level (1^{st} experiment)

Table 13. Armyworm assay of OsCRK6 transgenic rice under laboratory screen condition at construct level (2^{nd} experiment)

Table 14. Rice stem borer assay of OsCRK6 transgenic rice under greenhouse screen condition at line level

Table 15. Asian corn borer assay of OsMFS5 transgenic rice under laboratory screening condition at line level (1^{st} experiment)

Table 16. Asian corn borer assay of OsMFS5 transgenic rice under laboratory screening condition at line level (2^{nd} experiment)

Table 17. Armyworm assay of OsMFS5 transgenic rice under laboratory screen condition at line level (1^{st} experiment)

Table 18. OAW assay of OsMFS5 transgenic rice plants under laboratory screen condition at line level (2^{nd} experiment)

Table 19. Rice stem borer assay of OsMFS5 transgenic rice plants under greenhouse screen condition (withered rate)

Table 20. Rice stem borer assay of OsMFS5 transgenic rice plants under greenhouse screen condition (dead rate)

TABLE 1
SEQ ID NOs for nucleotide and amino acid
sequences provided in the sequence listing

| | | SEQ ID NO: | SEQ ID NO: |
|---|---|---|---|
| Source species | Clone Designation | (Nucleotide) | (Amino Acid) |
| Oryza sativa | T-DNA flanking | 1 | n/a |
| | sequence in | | |
| | AH43610 (RB) | | |
| Oryza sativa | T-DNA flanking | 2 | n/a |
| | sequence in | | |
| | AH43610 (LB) | | |
| Oryza sativa | T-DNA flanking | 11 | n/a |
| | sequence in | | |
| | AH43610 (RB) | | |
| Artificial sequence | DP0158 vector | 3 | n/a |
| Oryza sativa | OsCRK6 | 4, 5 | 6 |
| Oryza sativa | OsMFS5 | 12, 13 | 14 |
| Artificial | Primers | 7-10, 15-18 | n/a |

The sequence descriptions and Sequence Listing attached hereto comply with the rules governing nucleotide and/or amino acid sequence disclosures in patent applications as set forth in 37 C.F.R. § 1.821-1.825. The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (2):345-373 (1984) which are herein incorporated by reference. The symbols and format used for nucleotide and amino acid sequence data comply with the rules set forth in 37 C.F.R. § 1.822.

SEQ ID NO: 1 is the nucleotide sequence of flanking sequence of the inserted T-DNA at the right-border (RB) in AH43610 line.

SEQ ID NO: 2 is the nucleotide sequence of flanking sequence of the inserted T-DNA at the left-border (LB) in AH43610 line.

SEQ ID NO: 3 is the nucleotide sequence of vector DP0158.

SEQ ID NO: 4 is the nucleotide sequence of cDNA of OsCRK6 gene.

SEQ ID NO: 5 is the nucleotide sequence of CDS of OsCRK6 gene.

SEQ ID NO: 6 is the amino acid sequence of OsCRK6.

SEQ ID NO: 7 is forward primer for cloning cDNA of OsCRK6 gene.

SEQ ID NO: 8 is reverse primer for cloning cDNA of OsCRK6 gene.

SEQ ID NO: 9 is forward primer for real-time PCR analysis of OsCRK6 gene.

SEQ ID NO: 10 is reverse primer for real-time PCR analysis of OsCRK6 gene

SEQ ID NO: 11 is the nucleotide sequence of flanking sequence of the inserted T-DNA at the right-border (RB) in AH29691 line.

SEQ ID NO: 12 is the nucleotide sequence of cDNA of OsMFS5 gene.

SEQ ID NO: 13 is the nucleotide sequence of CDS of OsMFS5 gene.

SEQ ID NO: 14 is the amino acid sequence of OsMFS5.

SEQ ID NO: 15 is forward primer for cloning cDNA of OsMFS5 gene.

SEQ ID NO: 16 is reverse primer for cloning cDNA of OsMFS5 gene.

SEQ ID NO: 17 is forward primer for real-time PCR analysis of OsMFS5 gene.

SEQ ID NO: 18 is reverse primer for real-time PCR analysis of OsMFS5 gene.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants; reference to “a cell” includes one or more cells and equivalents thereof known to those skilled in the art, and so forth.

As used herein:

The term “OsCRK6” is a cysteine-rich receptor-like protein kinase 6 and refers to a rice polypeptide that confers increased tolerance to an insect pest and is encoded by the rice gene locus LOC_Os03g16960.1. “CRK6 polypeptide” refers herein to the OsCRK6 polypeptide and its homologs from other organisms.

The OsCRK6 polypeptide (SEQ ID NO: 6) is encoded by the coding sequence (CDS) (SEQ ID NO: 5) or nucleotide sequence (SEQ ID NO: 4) at rice gene locus LOC_Os03g16960.1. This polypeptide is annotated as “cysteine-rich repeat secretory protein 55 precursor, putative, expressed” in TIGR (the internet at plant biology msu.edu/index.shtml), however does not have any prior assigned function.

The term “OsMFS5” is a major facilitator superfamily 5 protein and refers to a rice polypeptide that confers increased tolerance to an insect pest and is encoded by the rice gene locus LOC_Os09g36600.1. “MFS5 polypeptide” refers herein to the OsMFS5 polypeptide and its homologs from other organisms.

The OsMFS5 polypeptide (SEQ ID NO: 14) is encoded by the coding sequence (CDS) (SEQ ID NO: 13) or nucleotide sequence (SEQ ID NO: 12) at rice gene locus LOC_Os09g36600.1. This polypeptide is annotated as “nodulin, putative, expressed” in TIGR (the internet at plant biology msu.edu/index.shtml), however does not have any prior assigned function.

The term “insect tolerance protein” is used herein to refer to a polypeptide that inhibits the growth of, stunts the growth of, and/or kills one or more insect pests, including, but not limited to, members of the Lepidoptera, Diptera, Hemiptera and Coleoptera orders.

The terms “monocot” and “monocotyledonous plant” are used interchangeably herein. A monocot of the current disclosure includes the Gramineae.

The terms “dicot” and “dicotyledonous plant” are used interchangeably herein. A dicot of the current disclosure includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

A “control” or “control plant” or “control plant cell” provides a reference point for measuring changes in phenotype of a subject plant or plant cell which was genetically altered by, such as transformation, and has been affected as to a gene of interest. A subject plant or plant cell may be descended from a plant or cell so altered and will comprise the alteration.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to a condition or stimulus that would induce expression of the gene of interest; or (e) the subject plant or plant cell itself, under conditions in which the gene of interest is not expressed.

In this disclosure, ZH11-TC and empty vector plants indicate control plants. ZH11-TC represents rice plants generated from tissue cultured Zhonghua 11, and empty vector represents plants transformed with empty vector DP0158.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Progeny” comprises any subsequent generation of a plant.

“Transgenic plant” includes reference to a plant which comprises within its genome a heterologous polynucleotide. The heterologous polynucleotide can be stably integrated within the genome such that the polynucleotide is passed on to successive generations. The heterologous polynucleotide may be integrated into the genome alone or as part of a recombinant DNA construct. A T₀ plant is directly recovered from the transformation and regeneration process. Progeny of T₀ plants are referred to as T₁ (first progeny generation), T₂ (second progeny generation), etc.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” refers to the RNA that is without introns and that can be translated into protein by the cell.

“cDNA” refers to a DNA that is complementary to and synthesized from an mRNA template using the enzyme reverse transcriptase. The cDNA can be single-stranded or converted into the double-stranded form using the Klenow fragment of DNA polymerase I.

“Mature” protein refers to a post-translationally processed polypeptide; i.e., one from which any pre- or pro-peptides present in the primary translation product has been removed.

“Precursor” protein refers to the primary product of translation of mRNA; i.e., with pre- and/or pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Non-genomic nucleic acid sequence” or “non-genomic nucleic acid molecule” or “non-genomic polynucleotide” refers to a nucleic acid molecule that has one or more change in the nucleic acid sequence compared to a native or genomic nucleic acid sequence. In some embodiments the change to a native or genomic nucleic acid molecule includes but is not limited to: changes in the nucleic acid sequence due to the degeneracy of the genetic code; codon optimization of the nucleic acid sequence for expression in plants; changes in the nucleic acid sequence to introduce at least one amino acid substitution, insertion, deletion and/or addition compared to the native or genomic sequence; removal of one or more intron associated with the genomic nucleic acid sequence; insertion of one or more heterologous introns; deletion of one or more upstream or downstream regulatory regions associated with the genomic nucleic acid sequence; insertion of one or more heterologous upstream or downstream regulatory regions; deletion of the 5′ and/or 3′ untranslated region associated with the genomic nucleic acid sequence; insertion of a heterologous 5′ and/or 3′ untranslated region; and modification of a polyadenylation site. In some embodiments the non-genomic nucleic acid molecule is a cDNA. In some embodiments the non-genomic nucleic acid molecule is a synthetic nucleic acid sequence.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature.

The terms “entry clone” and “entry vector” are used interchangeably herein.

“Regulatory sequences” and “regulatory elements” are used interchangeably and refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably and refer to a promoter that is expressed predominantly but not necessarily exclusively in one tissue or organ, but that may also be expressed in one specific cell.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

A “chloroplast transit peptide” is an amino acid sequence which is translated in conjunction with a protein and directs the protein to the chloroplast or other plastid types present in the cell in which the protein is made. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel (1992) Plant Phys. 100:1627-1632). A “mitochondrial signal peptide” is an amino acid sequence which directs a precursor protein into the mitochondria (Zhang and Glaser (2002) Trends Plant Sci 7:14-21).

Sequence alignments and percent identity calculations may be determined using a variety of comparison methods designed to detect homologous sequences including, but not limited to, the MEGALIGN® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp, CABIOS. 5:151-153 (1989)) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

Turning now to the embodiments:

Embodiments include isolated polynucleotides and polypeptides, recombinant DNA constructs useful for conferring insect tolerance, compositions (such as plants or seeds) comprising these recombinant DNA constructs, and methods utilizing these recombinant DNA constructs.

Isolated Polynucleotides and Polypeptides

The present disclosure includes the following isolated polynucleotides and polypeptides:

In some embodiments, polynucleotides are provided encoding CRK6 or MFS5 polypeptides.

In some embodiments, isolated polynucleotides are provided comprising: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO: 6 or 14; or (ii) a full complement of the nucleic acid sequence of (i), wherein the full complement and the nucleic acid sequence of (i) consist of the same number of nucleotides and are 100% complementary. Any of the foregoing isolated polynucleotides may be utilized in any recombinant DNA constructs of the present disclosure.

In some embodiments, isolated polypeptides are provided having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when compared to SEQ ID NO: 6 or 14. The polypeptides are insect tolerance polypeptide CRK6 or MFS5.

In some embodiments, isolated polynucleotide are provided comprising (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO: 4, 5, 12 or 13; or (ii) a full complement of the nucleic acid sequence of (i). Any of the foregoing isolated polynucleotides may be utilized in any recombinant DNA constructs of the present disclosure. The isolated polynucleotide preferably encodes an insect tolerance protein. Over-expression of this polypeptide increases plant tolerance to an insect pest.

Recombinant DNA Constructs

In one aspect, the present disclosure includes recombinant DNA constructs.

In one embodiment, a recombinant DNA construct comprises a polynucleotide operably linked to at least one regulatory sequence (e.g., a promoter functional in a plant), wherein the polynucleotide comprises (i) a nucleic acid sequence encoding an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO: 6 or 14; or (ii) a full complement of the nucleic acid sequence of (i).

In another embodiment, a recombinant DNA construct comprises a polynucleotide operably linked to at least one regulatory sequence (e.g., a promoter functional in a plant), wherein said polynucleotide comprises (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO: 4, 5, 12 or 13; or (ii) a full complement of the nucleic acid sequence of (i).

In another embodiment, a recombinant DNA construct comprises a polynucleotide operably linked to at least one regulatory sequence (e.g., a promoter functional in a plant), wherein said polynucleotide encodes a CRK6 or MFS5 protein. These polypeptides provide tolerance to an insect pest activity, and may be from, for example, Oryza sativa, Oryza australiensis, Oryza barthii, Oryza glaberrima (African rice), Oryza latifolia, Oryza longistaminata, Oryza meridionalis, Oryza officinalis, Oryza punctata, Oryza rufipogon (brownbeard or red rice), Oryza nivara (Indian wild rice), Arabidopsis thaliana, Zea mays, Glycine max, Glycine tabacina, Glycine soja or Glycine tomentella.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

“Suppression DNA construct” is a recombinant DNA construct which when transformed or stably integrated into the genome of the plant, results in “silencing” of a target gene in the plant. The target gene may be endogenous or transgenic to the plant. “Silencing”, as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, includes lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand) of the gene of interest.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as siRNA (short interfering RNA) constructs and miRNA (microRNA) constructs.

“Antisense inhibition” refers to the production of antisense RNA transcripts capable of suppressing the expression of the target gene or gene product. “Antisense RNA” refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence.

“Cosuppression” refers to the production of sense RNA transcripts capable of suppressing the expression of the target gene or gene product. “Sense” RNA refers to RNA transcript that includes the mRNA and can be translated into protein within a cell or in vitro. Cosuppression constructs in plants have been previously designed by focusing on over-expression of a nucleic acid sequence having homology to a native mRNA, in the sense orientation, which results in the reduction of all RNA having homology to the over-expressed sequence (see Vaucheret et al., Plant J. 16:651-659 (1998); and Gura, Nature 404:804-808 (2000)).

Another variation describes the use of plant viral sequences to direct the suppression of proximal mRNA encoding sequences (PCT Publication No. WO 98/36083 published on Aug. 20, 1998).

RNA interference (RNAi) refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes. Dev. 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

MicroRNAs (miRNAs) appear to regulate target genes by binding to complementary sequences located in the transcripts produced by these genes. It seems likely that miRNAs can enter at least two pathways of target gene regulation: (1) translational inhibition; and (2) RNA cleavage. MicroRNAs entering the RNA cleavage pathway are analogous to the 21-25 nt short interfering RNAs (siRNAs) generated during RNA interference (RNAi) in animals and posttranscriptional gene silencing (PTGS) in plants, and likely are incorporated into an RNA-induced silencing complex (RISC) that is similar or identical to that seen for RNAi.

Regulatory Sequences:

A recombinant DNA construct of the present disclosure may comprise at least one regulatory sequence.

A regulatory sequence may be a promoter or enhancer.

A number of promoters can be used in recombinant DNA constructs of the present disclosure. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 35S or UBI promoter may (or may not) have pleiotropic effects, although candidate gene efficacy may be estimated when driven by a constitutive promoter. Use of tissue-specific and/or stress-specific promoters may eliminate undesirable effects, but retain the ability to enhance insect tolerance. This type of effect has been observed in Arabidopsis for drought and cold tolerance (Kasuga et al., Nature Biotechnol. 17:287-91 (1999)).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the disclosure, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods of the present disclosure which causes the desired temporal and spatial expression.

Promoters which are seed or embryo-specific and may be useful in the disclosure include soybean Kunitz trypsin inhibitor (Kti3, Jofuku and Goldberg, Plant Cell 1:1079-1093 (1989)), patatin (potato tubers) (Rocha-Sosa, M., et al., EMBO J. 8:23-29 (1989)), convicilin, vicilin, and legumin (pea cotyledons) (Rerie, W. G., et al., Mol. Gen. Genet. 259:149-157 (1991); Newbigin, E. J., et al., Planta 180:461-470 (1990); Higgins, T. J. V., et al., Plant. Mol. Biol. 11:683-695 (1988)), zein (maize endosperm) (Schemthaner, J. P., et al., EMBO J. 7:1249-1255 (1988)), phaseolin (bean cotyledon) (Segupta-Gopalan, C., et al., Proc. Natl. Acad. Sci. U.S.A. 82:3320-3324 (1995)), phytohemagglutinin (bean cotyledon) (Voelker, T. et al., EMBO J. 6:3571-3577 (1987)), B-conglycinin and glycinin (soybean cotyledon) (Chen, Z-L, et al., EMBO J. 7:297-302 (1988)), glutelin (rice endosperm), hordein (barley endosperm) (Marris, C., et al., Plant Mol. Biol. 10:359-366 (1988)), glutenin and gliadin (wheat endosperm) (Colot, V., et al., EMBO J. 6:3559-3564 (1987)), and sporamin (sweet potato tuberous root) (Hattori, T., et al., Plant Mol. Biol. 14:595-604 (1990)). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove et al., Bio/Technology 7:L929-932 (1989)), bean lectin and bean beta-phaseolin promoters to express luciferase (Riggs et al., Plant Sci. 63:47-57 (1989)), and wheat glutenin promoters to express chloramphenicol acetyl transferase (Colot et al., EMBO J. 6:3559-3564 (1987)).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters for use in the current disclosure include the following: 1) the stress-inducible RD29A promoter (Kasuga et al., Nature Biotechnol. 17:287-91 (1999)); 2) the barley promoter, B22E; expression of B22E is specific to the pedicel in developing maize kernels (“Primary Structure of a Novel Barley Gene Differentially Expressed in Immature Aleurone Layers”, Klemsdal et al., Mol. Gen. Genet. 228(1/2):9-16 (1991)); and 3) maize promoter, Zag2 (“Identification and molecular characterization of ZAG1, the maize homolog of the Arabidopsis floral homeotic gene AGAMOUS”, Schmidt et al., Plant Cell 5(7):729-737 (1993); “Structural characterization, chromosomal localization and phylogenetic evaluation of two pairs of AGAMOUS-like MADS-box genes from maize”, Theissen et al., Gene 156(2):155-166 (1995); NCBI GenBank Accession No. X80206)). Zag2 transcripts can be detected five days prior to pollination to seven to eight days after pollination (“DAP”), and directs expression in the carpel of developing female inflorescences and CimI which is specific to the nucleus of developing maize kernels. CimI transcript is detected four to five days before pollination to six to eight DAP. Other useful promoters include any promoter which can be derived from a gene whose expression is maternally associated with developing female florets.

For the expression of a polynucleotide in developing seed tissue, promoters of particular interest include seed-preferred promoters, particularly early kernel/embryo promoters and late kernel/embryo promoters. Kernel development post-pollination is divided into approximately three primary phases. The lag phase of kernel growth occurs from about 0 to 10-12 DAP. During this phase the kernel is not growing significantly in mass, but rather important events are being carried out that will determine kernel vitality (e.g., number of cells established). The linear grain fill stage begins at about 10-12 DAP and continues to about 40 DAP. During this stage of kernel development, the kernel attains almost all of its final mass, and various storage products (i.e., starch, protein, oil) are produced. Finally, the maturation phase occurs from about 40 DAP to harvest. During this phase of kernel development the kernel becomes quiescent and begins to dry down in preparation for a long period of dormancy prior to germination. As defined herein “early kernel/embryo promoters” are promoters that drive expression principally in developing seed during the lag phase of development (i.e., from about 0 to about 12 DAP). “Late kernel/embryo promoters”, as defined herein, drive expression principally in developing seed from about 12 DAP through maturation. There may be some overlap in the window of expression. The choice of the promoter will depend on the ABA-associated sequence utilized and the phenotype desired.

Early kernel/embryo promoters include, for example, Cim1 that is active 5 DAP in particular tissues (WO 00/11177), which is herein incorporated by reference. Other early kernel/embryo promoters include the seed-preferred promoters end1 which is active 7-10 DAP, and end2, which is active 9-14 DAP in the whole kernel and active 10 DAP in the endosperm and pericarp (WO 00/12733), herein incorporated by reference. Additional early kernel/embryo promoters that find use in certain methods of the present disclosure include the seed-preferred promoter Itp2 (U.S. Pat. No. 5,525,716); maize Zm40 promoter (U.S. Pat. No. 6,403,862); maize nuc1c (U.S. Pat. No. 6,407,315); maize ckx1-2 promoter (U.S. Pat. No. 6,921,815 and US Patent Application Publication Number 2006/0037103); maize lec1 promoter (U.S. Pat. No. 7,122,658); maize ESR promoter (U.S. Pat. No. 7,276,596); maize ZAP promoter (U.S. Patent Application Publication Numbers 20040025206 and 20070136891); maize promoter eep1 (U.S. Patent Application Publication Number 20070169226); and maize promoter ADF4 (U.S. Patent Application No. 60/963,878, filed 7 Aug. 2007). Additional promoters for regulating the expression of the nucleotide sequences of the present disclosure in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

Promoters for use in the current disclosure may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (GenBank Accession No. EF030817), and the constitutive promoter GOS2 from Zea mays. Other promoters include root preferred promoters, such as the maize NAS2 promoter, the maize Cyclo promoter (US Publication No. 2006/0156439, published Jul. 13, 2006), the maize ROOTMET2 promoter (WO 2005/063998, published Jul. 14, 2005), the CR1BIO promoter (WO 2006/055487, published May 26, 2006), the CRWAQ81 promoter (WO 2005/035770, published Apr. 21, 2005) and the maize ZRP2.47 promoter (NCBI Accession No. U38790; NCBI GI No. 1063664).

Recombinant DNA constructs of the present disclosure may also include other regulatory sequences including, but not limited to, translation leader sequences, introns, and polyadenylation recognition sequences. In another embodiment of the present disclosure, a recombinant DNA construct of the present disclosure further comprises an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, Mol. Cell Biol. 8:4395-4405 (1988); Callis et al., Genes Dev. 1:1183-1200 (1987)).

An enhancer or enhancer element refers to a cis-acting transcriptional regulatory element, a.k.a. cis-element, which confers an aspect of the overall expression pattern, but is usually insufficient alone to drive transcription, of an operably linked polynucleotide sequence. An isolated enhancer element may be fused to a promoter to produce a chimeric promoter cis-element, which confers an aspect of the overall modulation of gene expression. Enhancers are known in the art and include the SV40 enhancer region, the CaMV 35S enhancer element, and the like. Some enhancers are also known to alter normal regulatory element expression patterns, for example, by causing a regulatory element to be expressed constitutively when without the enhancer, the same regulatory element is expressed only in one specific tissue or a few specific tissues. Duplicating the upstream region of the CaMV35S promoter has been shown to increase expression by approximately tenfold (Kay, R. et al., (1987) Science 236: 1299-1302).

Enhancers for use in the current disclosure may include CaMV 35S (Benfey, et al., (1990) EMBO J. 9:1685-96); 4×B3 P-CaMV.35S Enhancer Domain—four tandem copies of the B3 domain (208 to 155) as described in U.S. Pat. No. 5,097,025; 4×AS-1 P-CaMV.35S Enhancer Domain—four tandem copies of the “activation sequence” (83 to 62) as described in U.S. Pat. No. 5,097,025; 2×B1-B2 P-CaMV.35S Enhancer Domain—two tandem copies of the B1-B2 domain (148 to 90) as described in U.S. Pat. No. 5,097,025; 2×A1-B3 P-CaMV.35S Enhancer Domain—two tandem copies of the A1-B3 domain (208 to 46) as described in U.S. Pat. No. 5,097,025; 2×61-B5 P-CaMV.35S Enhancer Domain—two tandem copies of the B1-B5 domain (343 to 90) as described in U.S. Pat. No. 5,097,025; the omega enhancer or the omega prime enhancer (Gallie, et al., (1989) Molecular Biology of RNA ed. Cech (Liss, New York) 237-256 and Gallie, et al., (1987) Gene 60:217-25), the enhancers of U.S. Pat. No. 7,803,992, the sugarcane bacilliform viral (SCBV) enhancer element (WO2013130813).

Any plant can be selected for the identification of regulatory sequences and genes to be used in recombinant DNA constructs of the present disclosure. Examples of suitable plant targets for the isolation of genes and regulatory sequences would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castor bean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, maize, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugar beet, sugarcane, sunflower, sweet potato, sweet gum, tangerine, tea, tobacco, tomato, triticale, turf, turnip, a vine, watermelon, wheat, yams, and zucchini.

Compositions

A composition of the present disclosure is a plant comprising in its genome any of the recombinant DNA constructs of the present disclosure (such as any of the constructs discussed above). Compositions also include any progeny of the plant, and any seed obtained from the plant or its progeny, wherein the progeny or seed comprises within its genome the recombinant DNA construct. Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced recombinant DNA construct. These seeds can be grown to produce plants that would exhibit an altered agronomic characteristic, or used in a breeding program to produce hybrid seed, which can be grown to produce plants that would exhibit such an altered agronomic characteristic. The seeds may be maize seeds, or rice seeds.

The plant may be a monocotyledonous or dicotyledonous plant, for example, a maize or soybean plant, such as a maize hybrid plant or a maize inbred plant. The plant may also be sunflower, sorghum, canola, wheat, alfalfa, cotton, rice, barley or millet.

The recombinant DNA construct is stably integrated into the genome of the plant.

Embodiments include but are not limited to the following:

1. A transgenic plant (for example, a rice, maize or soybean plant) comprising in its genome a recombinant DNA construct comprising a polynucleotide operably linked to at least one heterologous regulatory sequence, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO: 6 or 14; and wherein said transgenic plant exhibits increased tolerance to an insect pest when compared to a control plant not comprising said recombinant DNA construct.

2. The transgenic plant of embodiment 1, wherein the polynucleotide encodes a CRK6 or MFS5 polypeptide, for example, from Oryza sativa, Oryza australiensis, Oryza barthii, Oryza glaberrima (African rice), Oryza latifolia, Oryza longistaminata, Oryza meridionalis, Oryza officinalis, Oryza punctata, Oryza rufipogon (brownbeard or red rice), Oryza nivara (Indian wild rice), Arabidopsis thaliana, Cicer arietinum, Solanum tuberosum, Brassica oleracea, Zea mays, Glycine max, Glycine tabacina, Glycine soja or Glycine tomentella.

3. The transgenic plant of any one of embodiments 1 to 2, wherein the transgenic plant further comprises at least one polynucleotide encoding an insecticidal polypeptide.

4. The transgenic plant of any one of embodiments 1 to 2, wherein the transgenic plant further comprises at least one recombinant polynucleotide encoding a polypeptide of interest.

5. Any progeny of the above plants in embodiments 1-4, any seeds of the above plants in embodiments 1-4, any seeds of progeny of the above plants in embodiments 1-4, and cells from any of the above plants in embodiments 1-4 and progeny thereof.

In any of the foregoing embodiments 1-5 or any other embodiments of the present disclosure, the recombinant DNA construct may comprises at least one heterologous promoter functional in a plant as a regulatory sequence.

By “insecticidal protein” is used herein to refer to a polypeptide that has toxic activity against one or more insect pests, including, but not limited to, members of the Lepidoptera, Diptera, Hemiptera and Coleoptera orders or the Nematoda phylum or a protein that has homology to such a protein. Pesticidal proteins have been isolated from organisms including, for example, Bacillus sp., Pseudomonas sp., Photorhabdus sp., Xenorhabdus sp., Clostridium bifermentans and Paenibacillus popilliae. Pesticidal proteins include but are not limited to: insecticidal proteins from Pseudomonas sp. such as PSEEN3174 (Monalysin; (2011) PLoS Pathogens 7:1-13); from Pseudomonas protegens strain CHAO and Pf-5 (previously fluorescens) (Pechy-Tarr, (2008) Environmental Microbiology 10:2368-2386; GenBank Accession No. EU400157); from Pseudomonas Taiwanensis (Liu, et al., (2010) J. Agric. Food Chem., 58:12343-12349) and from Pseudomonas pseudoalcligenes (Zhang, et al., (2009) Annals of Microbiology 59:45-50 and Li, et al., (2007) Plant Cell Tiss. Organ Cult. 89:159-168); insecticidal proteins from Photorhabdus sp. and Xenorhabdus sp. (Hinchliffe, et al., (2010) The Open Toxicology Journal, 3:101-118 and Morgan, et al., (2001) Applied and Envir. Micro. 67:2062-2069); U.S. Pat. No. 6,048,838, and U.S. Pat. No. 6,379,946; a PIP-1 polypeptide of US publication number US2014008054; an AfIP-1A and/or AfIP-1B polypeptide of U.S. Ser. No. 13/800,233; a PHI-4 polypeptide of U.S. Ser. No. 13/839,702; and δ-endotoxins including, but not limited to, the Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry 51, Cry55, Cry56, Cry57, Cry58, Cry59, Cry60, Cry61, Cry62, Cry63, Cry64, Cry65, Cry66, Cry67, Cry68, Cry69, Cry70, Cry71 and Cry72 classes of δ-endotoxin genes and the B. thuringiensis cytolytic cyt1 and cyt2 genes. Members of these classes of B. thuringiensis insecticidal proteins include, but are not limited to Cry1Aa1 (Accession # AAA22353); Cry1Aa2 (Accession # Accession # AAA22552); Cry1Aa3 (Accession # BAA00257); Cry1Aa4 (Accession # CAA31886); Cry1Aa5 (Accession # BAA04468); Cry1Aa6 (Accession # AAA86265); Cry1Aa7 (Accession # AAD46139); Cry1Aa8 (Accession #126149); Cry1Aa9 (Accession # BAA77213); Cry1Aa10 (Accession # AAD55382); Cry1Aa11 (Accession # CAA70856); Cry1Aa12 (Accession # AAP80146); Cry1Aa13 (Accession # AAM44305); Cry1Aa14 (Accession # AAP40639); Cry1Aa15 (Accession # AAY66993); Cry1Aa16 (Accession # HQ439776); Cry1Aa17 (Accession # HQ439788); Cry1Aa18 (Accession # HQ439790); Cry1Aa19 (Accession # HQ685121); Cry1Aa20 (Accession # JF340156); Cry1Aa21 (Accession # JN651496); Cry1Aa22 (Accession # KC158223); Cry1Ab1 (Accession # AAA22330); Cry1Ab2 (Accession # AAA22613); Cry1Ab3 (Accession # AAA22561); Cry1Ab4 (Accession # BAA00071); Cry1Ab5 (Accession # CAA28405); Cry1Ab6 (Accession # AAA22420); Cry1Ab7 (Accession # CAA31620); Cry1Ab8 (Accession # AAA22551); Cry1Ab9 (Accession # CAA38701); Cry1Ab10 (Accession # A29125); Cry1Ab11 (Accession #112419); Cry1Ab12 (Accession # AAC64003); Cry1Ab13 (Accession # AAN76494); Cry1Ab14 (Accession # AAG16877); Cry1Ab15 (Accession # AAO13302); Cry1Ab16 (Accession # AAK55546); Cry1Ab17 (Accession # AAT46415); Cry1Ab18 (Accession # AAQ88259); Cry1Ab19 (Accession # AAW31761); Cry1Ab20 (Accession # ABB72460); Cry1Ab21 (Accession # ABS18384); Cry1Ab22 (Accession # ABW87320); Cry1Ab23 (Accession # HQ439777); Cry1Ab24 (Accession # HQ439778); Cry1Ab25 (Accession # HQ685122); Cry1Ab26 (Accession # HQ847729); Cry1Ab27 (Accession # JN135249); Cry1Ab28 (Accession # JN135250); Cry1Ab29 (Accession # JN135251); Cry1Ab30 (Accession # JN135252); Cry1Ab31 (Accession # JN135253); Cry1Ab32 (Accession # JN135254); Cry1Ab33 (Accession # AAS93798); Cry1Ab34 (Accession # KC156668); Cry1Ab-like (Accession # AAK14336); Cry1Ab-like (Accession # AAK14337); Cry1Ab-like (Accession # AAK14338); Cry1Ab-like (Accession # ABG88858); Cry1Ac1 (Accession # AAA22331); Cry1Ac2 (Accession # AAA22338); Cry1Ac3 (Accession # CAA38098); Cry1Ac4 (Accession # AAA73077); Cry1Ac5 (Accession # AAA22339); Cry1Ac6 (Accession # AAA86266); Cry1Ac7 (Accession # AAB46989); Cry1Ac8 (Accession # AAC44841); Cry1Ac9 (Accession # AAB49768); Cry1Ac10 (Accession # CAA05505); Cry1Ac11 (Accession # CAA10270); Cry1Ac12 (Accession #112418); Cry1Ac13 (Accession # AAD38701); Cry1Ac14 (Accession # AAQ06607); Cry1Ac15 (Accession # AAN07788); Cry1Ac16 (Accession # AAU87037); Cry1Ac17 (Accession # AAX18704); Cry1Ac18 (Accession # AAY88347); Cry1Ac19 (Accession # ABD37053); Cry1Ac20 (Accession # ABB89046); Cry1Ac21 (Accession # AAY66992); Cry1Ac22 (Accession # ABZ01836); Cry1Ac23 (Accession # CAQ30431); Cry1Ac24 (Accession # ABL01535); Cry1Ac25 (Accession # FJ513324); Cry1Ac26 (Accession # FJ617446); Cry1Ac27 (Accession # FJ617447); Cry1Ac28 (Accession # ACM90319); Cry1Ac29 (Accession # DQ438941); Cry1Ac30 (Accession # GQ227507); Cry1Ac31 (Accession # GU446674); Cry1Ac32 (Accession # HM061081); Cry1Ac33 (Accession # GQ866913); Cry1Ac34 (Accession # HQ230364); Cry1Ac35 (Accession # JF340157); Cry1Ac36 (Accession # JN387137); Cry1Ac37 (Accession # JQ317685); Cry1Ad1 (Accession # AAA22340); Cry1Ad2 (Accession # CAA01880); Cry1Ae1 (Accession # AAA22410); Cry1Af1 (Accession # AAB82749); Cry1Ag1 (Accession # AAD46137); Cry1Ah1 (Accession # AAQ14326); Cry1Ah2 (Accession # ABB76664); Cry1Ah3 (Accession # HQ439779); Cry1Ai1 (Accession # AAO39719); Cry1Ai2 (Accession # HQ439780); Cry1A-like (Accession # AAK14339); Cry1Ba1 (Accession # CAA29898); Cry1Ba2 (Accession # CAA65003); Cry1Ba3 (Accession # AAK63251); Cry1Ba4 (Accession # AAK51084); Cry1Ba5 (Accession # AB020894); Cry1Ba6 (Accession # ABL60921); Cry1Ba7 (Accession # HQ439781); Cry1Bb1 (Accession # AAA22344); Cry1Bb2 (Accession # HQ439782); Cry1Bc1 (Accession # CAA86568); Cry1Bd1 (Accession # AAD10292); Cry1Bd2 (Accession # AAM93496); Cry1Be1 (Accession # AAC32850); Cry1Be2 (Accession # AAQ52387); Cry1Be3 (Accession # ACV96720); Cry1Be4 (Accession # HM070026); Cry1Bf1 (Accession # CAC50778); Cry1Bf2 (Accession # AAQ52380); Cry1Bg1 (Accession # AAO39720); Cry1Bh1 (Accession # HQ589331); Cry1Bi1 (Accession # KC156700); Cry1Ca1 (Accession # CAA30396); Cry1Ca2 (Accession # CAA31951); Cry1Ca3 (Accession # AAA22343); Cry1Ca4 (Accession # CAA01886); Cry1Ca5 (Accession # CAA65457); Cry1Ca6 [1] (Accession # AAF37224); Cry1Ca7 (Accession # AAG50438); Cry1Ca8 (Accession # AAM00264); Cry1Ca9 (Accession # AAL79362); Cry1Ca10 (Accession # AAN16462); Cry1Ca11 (Accession # AAX53094); Cry1Ca12 (Accession # HM070027); Cry1Ca13 (Accession # HQ412621); Cry1Ca14 (Accession # JN651493); Cry1Cb1 (Accession # M97880); Cry1Cb2 (Accession # AAG35409); Cry1Cb3 (Accession # ACD50894); Cry1Cb-like (Accession # AAX63901); Cry1Da1 (Accession # CAA38099); Cry1Da2 (Accession #176415); Cry1Da3 (Accession # HQ439784); Cry1Db1 (Accession # CAA80234); Cry1Db2 (Accession # AAK48937); Cry1Dc1 (Accession # ABK35074); Cry1Ea1 (Accession # CAA37933); Cry1Ea2 (Accession # CAA39609); Cry1Ea3 (Accession # AAA22345); Cry1Ea4 (Accession # AAD04732); Cry1Ea5 (Accession # A15535); Cry1Ea6 (Accession # AAL50330); Cry1Ea7 (Accession # AAW72936); Cry1Ea8 (Accession # ABX11258); Cry1Ea9 (Accession # HQ439785); Cry1Ea10 (Accession # ADR00398); Cry1Ea11 (Accession # JQ652456); Cry1Eb1 (Accession # AAA22346); Cry1Fa1 (Accession # AAA22348); Cry1Fa2 (Accession # AAA22347); Cry1Fa3 (Accession # HM070028); Cry1Fa4 (Accession # HM439638); Cry1Fb1 (Accession # CAA80235); Cry1Fb2 (Accession # BAA25298); Cry1Fb3 (Accession # AAF21767); Cry1Fb4 (Accession # AAC10641); Cry1Fb5 (Accession # AAO13295); Cry1Fb6 (Accession # ACD50892); Cry1Fb7 (Accession # ACD50893); Cry1Ga1 (Accession # CAA80233); Cry1Ga2 (Accession # CAA70506); Cry1Gb1 (Accession # AAD10291); Cry1Gb2 (Accession # AAO13756); Cry1Gc1 (Accession # AAQ52381); Cry1Ha1 (Accession # CAA80236); Cry1Hb1 (Accession # AAA79694); Cry1Hb2 (Accession # HQ439786); Cry1H-like (Accession # AAF01213); Cry1Ia1 (Accession # CAA44633); Cry1Ia2 (Accession # AAA22354); Cry1Ia3 (Accession # AAC36999); Cry1Ia4 (Accession # AAB00958); Cry1Ia5 (Accession # CAA70124); Cry1Ia6 (Accession # AAC26910); Cry1Ia7 (Accession # AAM73516); Cry1Ia8 (Accession # AAK66742); Cry1Ia9 (Accession # AAQ08616); Cry1Ia10 (Accession # AAP86782); Cry1Ia11 (Accession # CAC85964); Cry1Ia12 (Accession # AAV53390); Cry1Ia13 (Accession # ABF83202); Cry1Ia14 (Accession # ACG63871); Cry1Ia15 (Accession # FJ617445); Cry1Ia16 (Accession # FJ617448); Cry1Ia17 (Accession # GU989199); Cry1Ia18 (Accession # ADK23801); Cry1Ia19 (Accession # HQ439787); Cry1Ia20 (Accession # JQ228426); Cry1Ia21 (Accession # JQ228424); Cry1Ia22 (Accession # JQ228427); Cry1Ia23 (Accession # JQ228428); Cry1Ia24 (Accession # JQ228429); Cry1Ia25 (Accession # JQ228430); Cry1Ia26 (Accession # JQ228431); Cry1Ia27 (Accession # JQ228432); Cry1Ia28 (Accession # JQ228433); Cry1Ia29 (Accession # JQ228434); Cry11a30 (Accession # JQ317686); Cry1Ia31 (Accession # JX944038); Cry1Ia32 (Accession # JX944039); Cry1Ia33 (Accession # JX944040); Cry1Ib1 (Accession # AAA82114); Cry1Ib2 (Accession # ABW88019); Cry1Ib3 (Accession # ACD75515); Cry1Ib4 (Accession # HM051227); Cry1Ib5 (Accession # HM070028); Cry1Ib6 (Accession # ADK38579); Cry1Ib7 (Accession # JN571740); Cry1Ib8 (Accession # JN675714); Cry1Ib9 (Accession # JN675715); Cry1Ib10 (Accession # JN675716); Cry1Ib11 (Accession # JQ228423); Cry1Ic1 (Accession # AAC62933); Cry1Ic2 (Accession # AAE71691); Cry1Id1 (Accession # AAD44366); Cry1Id2 (Accession # JQ228422); Cry1Ie1 (Accession # AAG43526); Cry1Ie2 (Accession # HM439636); Cry1Ie3 (Accession # KC156647); Cry1Ie4 (Accession # KC156681); Cry1If1 (Accession # AAQ52382); Cry1Ig1 (Accession # KC156701); Cry1I-like (Accession # AAC31094); Cry1I-like (Accession # ABG88859); Cry1Ja1 (Accession # AAA22341); Cry1Ja2 (Accession # HM070030); Cry1Ja3 (Accession # JQ228425); Cry1Jb1 (Accession # AAA98959); Cry1Jc1 (Accession # AAC31092); Cry1Jc2 (Accession # AAQ52372); Cry1Jd1 (Accession # CAC50779); Cry1Ka1 (Accession # AAB00376); Cry1Ka2 (Accession # HQ439783); Cry1La1 (Accession # AAS60191); Cry1La2 (Accession # HM070031); Cry1Ma1 (Accession # FJ884067); Cry1Ma2 (Accession # KC156659); Cry1Na1 (Accession # KC156648); Cry1Nb1 (Accession # KC156678); Cry1-like (Accession # AAC31091); Cry2Aa1 (Accession # AAA22335); Cry2Aa2 (Accession # AAA83516); Cry2Aa3 (Accession # D86064); Cry2Aa4 (Accession # AAC04867); Cry2Aa5 (Accession # CAA10671); Cry2Aa6 (Accession # CAA10672); Cry2Aa7 (Accession # CAA10670); Cry2Aa8 (Accession # AAO13734); Cry2Aa9 (Accession # AAO13750); Cry2Aa10 (Accession # AAQ04263); Cry2Aa11 (Accession # AAQ52384); Cry2Aa12 (Accession # ABI83671); Cry2Aa13 (Accession # ABL01536); Cry2Aa14 (Accession # ACF04939); Cry2Aa15 (Accession # JN426947); Cry2Ab1 (Accession # AAA22342); Cry2Ab2 (Accession # CAA39075); Cry2Ab3 (Accession # AAG36762); Cry2Ab4 (Accession # AAO13296); Cry2Ab5 (Accession # AAQ04609); Cry2Ab6 (Accession # AAP59457); Cry2Ab7 (Accession # AAZ66347); Cry2Ab8 (Accession # ABC95996); Cry2Ab9 (Accession # ABC74968); Cry2Ab10 (Accession # EF157306); Cry2Ab11 (Accession # CAM84575); Cry2Ab12 (Accession # ABM21764); Cry2Ab13 (Accession # ACG76120); Cry2Ab14 (Accession # ACG76121); Cry2Ab15 (Accession # HM037126); Cry2Ab16 (Accession # GQ866914); Cry2Ab17 (Accession # HQ439789); Cry2Ab18 (Accession # JN135255); Cry2Ab19 (Accession # JN135256); Cry2Ab20 (Accession # JN135257); Cry2Ab21 (Accession # JN135258); Cry2Ab22 (Accession # JN135259); Cry2Ab23 (Accession # JN135260); Cry2Ab24 (Accession # JN135261); Cry2Ab25 (Accession # JN415485); Cry2Ab26 (Accession # JN426946); Cry2Ab27 (Accession # JN415764); Cry2Ab28 (Accession # JN651494); Cry2Ac1 (Accession # CAA40536); Cry2Ac2 (Accession # AAG35410); Cry2Ac3 (Accession # AAQ52385); Cry2Ac4 (Accession # ABC95997); Cry2Ac5 (Accession # ABC74969); Cry2Ac6 (Accession # ABC74793); Cry2Ac7 (Accession # CAL18690); Cry2Ac8 (Accession # CAM09325); Cry2Ac9 (Accession # CAM09326); Cry2Ac10 (Accession # ABN15104); Cry2Ac11 (Accession # CAM83895); Cry2Ac12 (Accession # CAM83896); Cry2Ad1 (Accession # AAF09583); Cry2Ad2 (Accession # ABC86927); Cry2Ad3 (Accession # CAK29504); Cry2Ad4 (Accession # CAM32331); Cry2Ad5 (Accession # CA078739); Cry2Ae1 (Accession # AAQ52362); Cry2Af1 (Accession # AB030519); Cry2Af2 (Accession # GQ866915); Cry2Ag1 (Accession # ACH91610); Cry2Ah1 (Accession # EU939453); Cry2Ah2 (Accession # ACL80665); Cry2Ah3 (Accession # GU073380); Cry2Ah4 (Accession # KC156702); Cry2Ai1 (Accession # FJ788388); Cry2Aj (Accession #); Cry2Ak1 (Accession # KC156660); Cry2Ba1 (Accession # KC156658); Cry3Aa1 (Accession # AAA22336); Cry3Aa2 (Accession # AAA22541); Cry3Aa3 (Accession # CAA68482); Cry3Aa4 (Accession # AAA22542); Cry3Aa5 (Accession # AAA50255); Cry3Aa6 (Accession # AAC43266); Cry3Aa7 (Accession # CAB41411); Cry3Aa8 (Accession # AAS79487); Cry3Aa9 (Accession # AAW05659); Cry3Aa10 (Accession # AAU29411); Cry3Aa11 (Accession # AAW82872); Cry3Aa12 (Accession # ABY49136); Cry3Ba1 (Accession # CAA34983); Cry3Ba2 (Accession # CAA00645); Cry3Ba3 (Accession # JQ397327); Cry3Bb1 (Accession # AAA22334); Cry3Bb2 (Accession # AAA74198); Cry3Bb3 (Accession #115475); Cry3Ca1 (Accession # CAA42469); Cry4Aa1 (Accession # CAA68485); Cry4Aa2 (Accession # BAA00179); Cry4Aa3 (Accession # CAD30148); Cry4Aa4 (Accession # AFB18317); Cry4A-like (Accession # AAY96321); Cry4Ba1 (Accession # CAA30312); Cry4Ba2 (Accession # CAA30114); Cry4Ba3 (Accession # AAA22337); Cry4Ba4 (Accession # BAA00178); Cry4Ba5 (Accession # CAD30095); Cry4Ba-like (Accession # ABC47686); Cry4Ca1 (Accession # EU646202); Cry4Cb1 (Accession # FJ403208); Cry4Cb2 (Accession # FJ597622); Cry4Cc1 (Accession # FJ403207); Cry5Aa1 (Accession # AAA67694); Cry5Ab1 (Accession # AAA67693); Cry5Ac1 (Accession #134543); Cry5Ad1 (Accession # ABQ82087); Cry5Ba1 (Accession # AAA68598); Cry5Ba2 (Accession # ABW88931); Cry5Ba3 (Accession # AFJ04417); Cry5Ca1 (Accession # HM461869); Cry5Ca2 (Accession # ZP_04123426); Cry5Da1 (Accession # HM461870); Cry5Da2 (Accession # ZP_04123980); Cry5Ea1 (Accession # HM485580); Cry5Ea2 (Accession # ZP_04124038); Cry6Aa1 (Accession # AAA22357); Cry6Aa2 (Accession # AAM46849); Cry6Aa3 (Accession # ABH03377); Cry6Ba1 (Accession # AAA22358); Cry7Aa1 (Accession # AAA22351); Cry7Ab1 (Accession # AAA21120); Cry7Ab2 (Accession # AAA21121); Cry7Ab3 (Accession # ABX24522); Cry7Ab4 (Accession # EU380678); Cry7Ab5 (Accession # ABX79555); Cry7Ab6 (Accession # AC144005); Cry7Ab7 (Accession # ADB89216); Cry7Ab8 (Accession # GU145299); Cry7Ab9 (Accession # ADD92572); Cry7Ba1 (Accession # ABB70817); Cry7Bb1 (Accession # KC156653); Cry7Ca1 (Accession # ABR67863); Cry7Cb1 (Accession # KC156698); Cry7Da1 (Accession # ACQ99547); Cry7Da2 (Accession # HM572236); Cry7Da3 (Accession # KC156679); Cry7Ea1 (Accession # HM035086); Cry7Ea2 (Accession # HM132124); Cry7Ea3 (Accession # EEM19403); Cry7Fa1 (Accession # HM035088); Cry7Fa2 (Accession # EEM19090); Cry7Fb1 (Accession # HM572235); Cry7Fb2 (Accession # KC156682); Cry7Ga1 (Accession # HM572237); Cry7Ga2 (Accession # KC156669); Cry7Gb1 (Accession # KC156650); Cry7Gc1 (Accession # KC156654); Cry7Gd1 (Accession # KC156697); Cry7Ha1 (Accession # KC156651); Cry71a1 (Accession # KC156665); Cry7Ja1 (Accession # KC156671); Cry7Ka1 (Accession # KC156680); Cry7Kb1 (Accession # BAM99306); Cry7La1 (Accession # BAM99307); Cry8Aa1 (Accession # AAA21117); Cry8Ab1 (Accession # EU044830); Cry8Ac1 (Accession # KC156662); Cry8Ad1 (Accession # KC156684); Cry8Ba1 (Accession # AAA21118); Cry8Bb1 (Accession # CAD57542); Cry8Bc1 (Accession # CAD57543); Cry8Ca1 (Accession # AAA21119); Cry8Ca2 (Accession # AAR98783); Cry8Ca3 (Accession # EU625349); Cry8Ca4 (Accession # ADB54826); Cry8Da1 (Accession # BAC07226); Cry8Da2 (Accession # BD133574); Cry8Da3 (Accession # BD133575); Cry8Db1 (Accession # BAF93483); Cry8Ea1 (Accession # AAQ73470); Cry8Ea2 (Accession # EU047597); Cry8Ea3 (Accession # KC855216); Cry8Fa1 (Accession # AAT48690); Cry8Fa2 (Accession # HQ174208); Cry8Fa3 (Accession # AFH78109); Cry8Ga1 (Accession # AAT46073); Cry8Ga2 (Accession # ABC42043); Cry8Ga3 (Accession # FJ198072); Cry8Ha1 (Accession # AAW81032); Cry81a1 (Accession # EU381044); Cry8Ia2 (Accession # GU073381); Cry8Ia3 (Accession # HM044664); Cry8Ia4 (Accession # KC156674); Cry81b1 (Accession # GU325772); Cry8Ib2 (Accession # KC156677); Cry8Ja1 (Accession # EU625348); Cry8Ka1 (Accession # FJ422558); Cry8Ka2 (Accession # ACN87262); Cry8Kb1 (Accession # HM123758); Cry8Kb2 (Accession # KC156675); Cry8La1 (Accession # GU325771); Cry8Ma1 (Accession # HM044665); Cry8Ma2 (Accession # EEM86551); Cry8Ma3 (Accession # HM210574); Cry8Na1 (Accession # HM640939); Cry8Pa1 (Accession # HQ388415); Cry8Qa1 (Accession # HQ441166); Cry8Qa2 (Accession # KC152468); Cry8Ra1 (Accession # AFP87548); Cry8Sa1 (Accession # JQ740599); Cry8Ta1 (Accession # KC156673); Cry8-like (Accession # FJ770571); Cry8-like (Accession # ABS53003); Cry9Aa1 (Accession # CAA41122); Cry9Aa2 (Accession # CAA41425); Cry9Aa3 (Accession # GQ249293); Cry9Aa4 (Accession # GQ249294); Cry9Aa5 (Accession # JX174110); Cry9Aa like (Accession # AAQ52376); Cry9Ba1 (Accession # CAA52927); Cry9Ba2 (Accession # GU299522); Cry9Bb1 (Accession # AAV28716); Cry9Ca1 (Accession # CAA85764); Cry9Ca2 (Accession # AAQ52375); Cry9Da1 (Accession # BAA19948); Cry9Da2 (Accession # AAB97923); Cry9Da3 (Accession # GQ249293); Cry9Da4 (Accession # GQ249297); Cry9Db1 (Accession # AAX78439); Cry9Dc1 (Accession # KC156683); Cry9Ea1 (Accession # BAA34908); Cry9Ea2 (Accession # AAO12908); Cry9Ea3 (Accession # ABM21765); Cry9Ea4 (Accession # ACE88267); Cry9Ea5 (Accession # ACF04743); Cry9Ea6 (Accession # ACG63872); Cry9Ea7 (Accession # FJ380927); Cry9Ea8 (Accession # GQ249292); Cry9Ea9 (Accession # JN651495); Cry9Eb1 (Accession # CAC50780); Cry9Eb2 (Accession # GQ249298); Cry9Eb3 (Accession # KC156646); Cry9Ec1 (Accession # AAC63366); Cry9Ed1 (Accession # AAX78440); Cry9Ee1 (Accession # GQ249296); Cry9Ee2 (Accession # KC156664); Cry9Fa1 (Accession # KC156692); Cry9Ga1 (Accession # KC156699); Cry9-like (Accession # AAC63366); Cry10Aa1 (Accession # AAA22614); Cry10Aa2 (Accession # E00614); Cry10Aa3 (Accession # CAD30098); Cry10Aa4 (Accession # AFB18318); Cry10A-like (Accession # DQ167578); Cry1Ma1 (Accession # AAA22352); Cry11Aa2 (Accession # AAA22611); Cry11Aa3 (Accession # CAD30081); Cry11Aa4 (Accession # AFB18319); Cry11Aa-like (Accession # DQ166531); Cry11Ba1 (Accession # CAA60504); Cry11 Bb1 (Accession # AAC97162); Cry11Bb2 (Accession # HM068615); Cry12Aa1 (Accession # AAA22355); Cry13Aa1 (Accession # AAA22356); Cry14Aa1 (Accession # AAA21516); Cry14Ab1 (Accession # KC156652); Cry15Aa1 (Accession # AAA22333); Cry16Aa1 (Accession # CAA63860); Cry17Aa1 (Accession # CAA67841); Cry18Aa1 (Accession # CAA67506); Cry18Ba1 (Accession # AAF89667); Cry18Ca1 (Accession # AAF89668); Cry19Aa1 (Accession # CAA68875); Cry19Ba1 (Accession # BAA32397); Cry19Ca1 (Accession # AFM37572); Cry20Aa1 (Accession # AAB93476); Cry20Ba1 (Accession # ACS93601); Cry20Ba2 (Accession # KC156694); Cry20-like (Accession # GQ144333); Cry21Aa1 (Accession #132932); Cry21Aa2 (Accession #166477); Cry21Ba1 (Accession # BAC06484); Cry21Ca1 (Accession # JF521577); Cry21Ca2 (Accession # KC156687); Cry21Da1 (Accession # JF521578); Cry22Aa1 (Accession #134547); Cry22Aa2 (Accession # CAD43579); Cry22Aa3 (Accession # ACD93211); Cry22Ab1 (Accession # AAK50456); Cry22Ab2 (Accession # CAD43577); Cry22Ba1 (Accession # CAD43578); Cry22Bb1 (Accession # KC156672); Cry23Aa1 (Accession # AAF76375); Cry24Aa1 (Accession # AAC61891); Cry24Ba1 (Accession # BAD32657); Cry24Ca1 (Accession # CAJ43600); Cry25Aa1 (Accession # AAC61892); Cry26Aa1 (Accession # AAD25075); Cry27Aa1 (Accession # BAA82796); Cry28Aa1 (Accession # AAD24189); Cry28Aa2 (Accession # AAG00235); Cry29Aa1 (Accession # CAC80985); Cry30Aa1 (Accession # CAC80986); Cry30Ba1 (Accession # BAD00052); Cry30Ca1 (Accession # BAD67157); Cry30Ca2 (Accession # ACU24781); Cry30Da1 (Accession # EF095955); Cry30Db1 (Accession # BAE80088); Cry30Ea1 (Accession # ACC95445); Cry30Ea2 (Accession # FJ499389); Cry30Fa1 (Accession # AC122625); Cry30Ga1 (Accession # ACG60020); Cry30Ga2 (Accession # HQ638217); Cry31Aa1 (Accession # BAB11757); Cry31Aa2 (Accession # AAL87458); Cry31Aa3 (Accession # BAE79808); Cry31Aa4 (Accession # BAF32571); Cry31Aa5 (Accession # BAF32572); Cry31Aa6 (Accession # BAI44026); Cry31Ab1 (Accession # BAE79809); Cry31Ab2 (Accession # BAF32570); Cry31Ac1 (Accession # BAF34368); Cry31Ac2 (Accession # AB731600); Cry31Ad1 (Accession # BAI44022); Cry32Aa1 (Accession # AAG36711); Cry32Aa2 (Accession # GU063849); Cry32Ab1 (Accession # GU063850); Cry32Ba1 (Accession # BAB78601); Cry32Ca1 (Accession # BAB78602); Cry32Cb1 (Accession # KC156708); Cry32Da1 (Accession # BAB78603); Cry32Ea1 (Accession # GU324274); Cry32Ea2 (Accession # KC156686); Cry32Eb1 (Accession # KC156663); Cry32Fa1 (Accession # KC156656); Cry32Ga1 (Accession # KC156657); Cry32Ha1 (Accession # KC156661); Cry32Hb1 (Accession # KC156666); Cry32Ia1 (Accession # KC156667); Cry32Ja1 (Accession # KC156685); Cry32Ka1 (Accession # KC156688); Cry32La1 (Accession # KC156689); Cry32Ma1 (Accession # KC156690); Cry32Mb1 (Accession # KC156704); Cry32Na1 (Accession # KC156691); Cry32Oa1 (Accession # KC156703); Cry32Pa1 (Accession # KC156705); Cry32Qa1 (Accession # KC156706); Cry32Ra1 (Accession # KC156707); Cry32Sa1 (Accession # KC156709); Cry32Ta1 (Accession # KC156710); Cry32Ua1 (Accession # KC156655); Cry33Aa1 (Accession # AAL26871); Cry34Aa1 (Accession # AAG50341); Cry34Aa2 (Accession # AAK64560); Cry34Aa3 (Accession # AAT29032); Cry34Aa4 (Accession # AAT29030); Cry34Ab1 (Accession # AAG41671); Cry34Ac1 (Accession # AAG50118); Cry34Ac2 (Accession # AAK64562); Cry34Ac3 (Accession # AAT29029); Cry34Ba1 (Accession # AAK64565); Cry34Ba2 (Accession # AAT29033); Cry34Ba3 (Accession # AAT29031); Cry35Aa1 (Accession # AAG50342); Cry35Aa2 (Accession # AAK64561); Cry35Aa3 (Accession # AAT29028); Cry35Aa4 (Accession # AAT29025); Cry35Ab1 (Accession # AAG41672); Cry35Ab2 (Accession # AAK64563); Cry35Ab3 (Accession # AY536891); Cry35Ac1 (Accession # AAG50117); Cry35Ba1 (Accession # AAK64566); Cry35Ba2 (Accession # AAT29027); Cry35Ba3 (Accession # AAT29026); Cry36Aa1 (Accession # AAK64558); Cry37Aa1 (Accession # AAF76376); Cry38Aa1 (Accession # AAK64559); Cry39Aa1 (Accession # BAB72016); Cry40Aa1 (Accession # BAB72018); Cry40Ba1 (Accession # BAC77648); Cry40Ca1 (Accession # EU381045); Cry40Da1 (Accession # ACF15199); Cry41Aa1 (Accession # BAD35157); Cry41Ab1 (Accession # BAD35163); Cry41Ba1 (Accession # HM461871); Cry41Ba2 (Accession # ZP_04099652); Cry42Aa1 (Accession # BAD35166); Cry43Aa1 (Accession # BAD15301); Cry43Aa2 (Accession # BAD95474); Cry43Ba1 (Accession # BAD15303); Cry43Ca1 (Accession # KC156676); Cry43Cb1 (Accession # KC156695); Cry43Cc1 (Accession # KC156696); Cry43-like (Accession # BAD15305); Cry44Aa (Accession # BAD08532); Cry45Aa (Accession # BAD22577); Cry46Aa (Accession # BAC79010); Cry46Aa2 (Accession # BAG68906); Cry46Ab (Accession # BAD35170); Cry47Aa (Accession # AAY24695); Cry48Aa (Accession # CAJ18351); Cry48Aa2 (Accession # CAJ86545); Cry48Aa3 (Accession # CAJ86546); Cry48Ab (Accession # CAJ86548); Cry48Ab2 (Accession # CAJ86549); Cry49Aa (Accession # CAH56541); Cry49Aa2 (Accession # CAJ86541); Cry49Aa3 (Accession # CAJ86543); Cry49Aa4 (Accession # CAJ86544); Cry49Ab1 (Accession # CAJ86542); Cry50Aa1 (Accession # BAE86999); Cry50Ba1 (Accession # GU446675); Cry50Ba2 (Accession # GU446676); Cry51Aa1 (Accession # ABI14444); Cry51Aa2 (Accession # GU570697); Cry52Aa1 (Accession # EF613489); Cry52Ba1 (Accession # FJ361760); Cry53Aa1 (Accession # EF633476); Cry53Ab1 (Accession # FJ361759); Cry54Aa1 (Accession # ACA52194); Cry54Aa2 (Accession # GQ140349); Cry54Ba1 (Accession # GU446677); Cry55Aa1 (Accession # ABW88932); Cry54Ab1 (Accession # JQ916908); Cry55Aa2 (Accession # AAE33526); Cry56Aa1 (Accession # ACU57499); Cry56Aa2 (Accession # GQ483512); Cry56Aa3 (Accession # JX025567); Cry57Aa1 (Accession # ANC87261); Cry58Aa1 (Accession # ANC87260); Cry59Ba1 (Accession # JN790647); Cry59Aa1 (Accession # ACR43758); Cry60Aa1 (Accession # ACU24782); Cry60Aa2 (Accession # EA057254); Cry60Aa3 (Accession # EEM99278); Cry60Ba1 (Accession # GU810818); Cry60Ba2 (Accession # EA057253); Cry60Ba3 (Accession # EEM99279); Cry61Aa1 (Accession # HM035087); Cry61Aa2 (Accession # HM132125); Cry61Aa3 (Accession # EEM19308); Cry62Aa1 (Accession # HM054509); Cry63Aa1 (Accession # BAI44028); Cry64Aa1 (Accession # BAJ05397); Cry65Aa1 (Accession # HM461868); Cry65Aa2 (Accession # ZP_04123838); Cry66Aa1 (Accession # HM485581); Cry66Aa2 (Accession # ZP_04099945); Cry67Aa1 (Accession # HM485582); Cry67Aa2 (Accession # ZP_04148882); Cry68Aa1 (Accession # HQ113114); Cry69Aa1 (Accession # HQ401006); Cry69Aa2 (Accession # JQ821388); Cry69Ab1 (Accession # JN209957); Cry70Aa1 (Accession # JN646781); Cry70Ba1 (Accession # AD051070); Cry70Bb1 (Accession # EEL67276); Cry71Aa1 (Accession # JX025568); Cry72Aa1 (Accession # JX025569); Cyt1Aa (GenBank Accession Number X03182); Cyt1Ab (GenBank Accession Number X98793); Cyt1B (GenBank Accession Number U37196); Cyt2A (GenBank Accession Number Z14147); and Cyt2B (GenBank Accession Number U52043).

Examples of δ-endotoxins also include but are not limited to Cry1A proteins of U.S. Pat. Nos. 5,880,275 and 7,858,849; a DIG-3 or DIG-11 toxin (N-terminal deletion of α-helix 1 and/or α-helix 2 variants of cry proteins such as Cry1A, Cry3A) of U.S. Pat. Nos. 8,304,604, 8,304,605 and 8,476,226; Cry1B of U.S. patent application Ser. No. 10/525,318; Cry1C of U.S. Pat. No. 6,033,874; Cry1F of U.S. Pat. Nos. 5,188,960 and 6,218,188; Cry1A/F chimeras of U.S. Pat. Nos. 7,070,982; 6,962,705 and 6,713,063; a Cry2 protein such as Cry2Ab protein of U.S. Pat. No. 7,064,249); a Cry3A protein including but not limited to an engineered hybrid insecticidal protein (eHIP) created by fusing unique combinations of variable regions and conserved blocks of at least two different Cry proteins (US Patent Application Publication Number 2010/0017914); a Cry4 protein; a Cry5 protein; a Cry6 protein; Cry8 proteins of U.S. Pat. Nos. 7,329,736, 7,449,552, 7,803,943, 7,476,781, 7,105,332, 7,378,499 and 7,462,760; a Cry9 protein such as members of the Cry9A, Cry9B, Cry9C, Cry9D, Cry9E and Cry9F families; a Cry15 protein of Naimov, et al., (2008) Applied and Environmental Microbiology, 74:7145-7151; a Cry22, a Cry34Ab1 protein of U.S. Pat. Nos. 6,127,180, 6,624,145 and 6,340,593; a CryET33 and cryET34 protein of U.S. Pat. Nos. 6,248,535, 6,326,351, 6,399,330, 6,949,626, 7,385,107 and 7,504,229; a CryET33 and CryET34 homologs of US Patent Publication Number 2006/0191034, 2012/0278954, and PCT Publication Number WO 2012/139004; a Cry35Ab1 protein of U.S. Pat. Nos. 6,083,499, 6,548,291 and 6,340,593; a Cry46 protein, a Cry 51 protein, a Cry binary toxin; a TIC901 or related toxin; TIC807 of US Patent Application Publication Number 2008/0295207; ET29, ET37, TIC809, TIC810, TIC812, TIC127, TIC128 of PCT US 2006/033867; AXMI-027, AXMI-036, and AXMI-038 of U.S. Pat. No. 8,236,757; AXMI-031, AXMI-039, AXMI-040, AXMI-049 of U.S. Pat. No. 7,923,602; AXMI-018, AXMI-020 and AXMI-021 of WO 2006/083891; AXMI-010 of WO 2005/038032; AXMI-003 of WO 2005/021585; AXMI-008 of US Patent Application Publication Number 2004/0250311; AXMI-006 of US Patent Application Publication Number 2004/0216186; AXMI-007 of US Patent Application Publication Number 2004/0210965; AXMI-009 of US Patent Application Number 2004/0210964; AXMI-014 of US Patent Application Publication Number 2004/0197917; AXMI-004 of US Patent Application Publication Number 2004/0197916; AXMI-028 and AXMI-029 of WO 2006/119457; AXMI-007, AXMI-008, AXMI-0080rf2, AXMI-009, AXMI-014 and AXMI-004 of WO 2004/074462; AXMI-150 of U.S. Pat. No. 8,084,416; AXMI-205 of US Patent Application Publication Number 2011/0023184; AXMI-011, AXMI-012, AXMI-013, AXMI-015, AXMI-019, AXMI-044, AXMI-037, AXMI-043, AXMI-033, AXMI-034, AXMI-022, AXMI-023, AXMI-041, AXMI-063 and AXMI-064 of US Patent Application Publication Number 2011/0263488; AXMI-R1 and related proteins of US Patent Application Publication Number 2010/0197592; AXMI221Z, AXMI222z, AXMI223z, AXMI224z and AXMI225z of WO 2011/103248; AXMI218, AXMI219, AXMI220, AXMI226, AXMI227, AXMI228, AXMI229, AXMI230 and AXMI231 of WO 2011/103247; AXMI-115, AXMI-113, AXMI-005, AXMI-163 and AXMI-184 of U.S. Pat. No. 8,334,431; AXMI-001, AXMI-002, AXMI-030, AXMI-035 and AXMI-045 of US Patent Application Publication Number 2010/0298211; AXMI-066 and AXMI-076 of US Patent Application Publication Number 2009/0144852; AXMI128, AXMI130, AXMI131, AXMI133, AXMI140, AXMI141, AXMI142, AXMI143, AXMI144, AXMI146, AXMI148, AXMI149, AXMI152, AXMI153, AXMI154, AXMI155, AXMI156, AXMI157, AXMI158, AXMI162, AXMI165, AXMI166, AXMI167, AXMI168, AXMI169, AXMI170, AXMI171, AXMI172, AXMI173, AXMI174, AXMI175, AXMI176, AXMI177, AXMI178, AXMI179, AXMI180, AXMI181, AXMI182, AXMI185, AXMI186, AXMI187, AXMI188, AXMI189 of U.S. Pat. No. 8,318,900; AXMI079, AXMI080, AXMI081, AXMI082, AXMI091, AXMI092, AXMI096, AXMI097, AXMI098, AXMI099, AXMI100, AXMI101, AXMI102, AXMI103, AXMI104, AXMHI107, AXMI108, AXMI109, AXMI110, AXMI111, AXMI112, AXMI114, AXMI116, AXMI117, AXMI118, AXMI119, AXMI120, AXMI121, AXMI122, AXMI123, AXMI124, AXMI1257, AXMI1268, AXMI127, AXMI129, AXMI164, AXMI151, AXMI161, AXMI183, AXMI132, AXMI138, AXMI137 of US Patent Application Publication Number 2010/0005543, AXMI232, AXMI233 and AXMI249 of US Patent Application Publication Number 201400962281; cry proteins such as Cry1A and Cry3A having modified proteolytic sites of U.S. Pat. No. 8,319,019; a Cry1Ac, Cry2Aa and Cry1Ca toxin protein from Bacillus thuringiensis strain VBTS 2528 of US Patent Application Publication Number 2011/0064710. Other Cry proteins are well known to one skilled in the art (see, Crickmore, et al., “Bacillus thuringiensis toxin nomenclature” (2011), at lifesci.sussex.ac.uk/home/Neil_Crickmore/Bt/ which can be accessed on the world-wide web using the “www” prefix). The insecticidal activity of Cry proteins is well known to one skilled in the art (for review, see, van Frannkenhuyzen, (2009) J. Invert. Path. 101:1-16). The use of Cry proteins as transgenic plant traits is well known to one skilled in the art and Cry-transgenic plants including but not limited to plants expressing Cry1Ac, Cry1Ac+Cry2Ab, Cry1Ab, Cry1A.105, Cry1F, Cry1Fa2, Cry1F+Cry1Ac, Cry2Ab, Cry3A, mCry3A, Cry3Bb1, Cry34Ab1, Cry35Ab1, Vip3A, Cry9c and CBI-Bt have received regulatory approval (see, Sanahuja, (2011) Plant Biotech Journal 9:283-300 and the CERA. (2010) GM Crop Database Center for Environmental Risk Assessment (CERA), ILSI Research Foundation, Washington D.C. at cera-gmc.org/index.php?action=gm_crop_database which can be accessed on the world-wide web using the “www” prefix). More than one pesticidal proteins well known to one skilled in the art can also be expressed in plants such as Vip3Ab & Cry1Fa (US2012/0317682); Cry1BE & Cry1F (US2012/0311746); Cry1CA & Cry1AB (US2012/0311745); Cry1F & CryCa (US2012/0317681); Cry1DA & Cry1BE (US2012/0331590); Cry1DA & Cry1Fa (US2012/0331589); Cry1AB & Cry1BE (US2012/0324606); Cry1Fa & Cry2Aa and Cry1I & Cry1E (US2012/0324605); Cry34Ab/35Ab and Cry6Aa (US20130167269); Cry34Ab/VCry35Ab & Cry3Aa (US20130167268); and Cry3A and Cry1Ab or Vip3Aa (US20130116170). Pesticidal proteins also include insecticidal lipases including lipid acyl hydrolases of U.S. Pat. No. 7,491,869, and cholesterol oxidases such as from Streptomyces (Purcell et al. (1993) Biochem Biophys Res Commun 15:1406-1413). Pesticidal proteins also include VIP (vegetative insecticidal proteins) toxins of U.S. Pat. Nos. 5,877,012, 6,107,279 6,137,033, 7,244,820, 7,615,686, and 8,237,020 and the like. Other VIP proteins are well known to one skilled in the art (see, lifesci.sussex.ac.uk/home/Neil_Crickmore/Bt/vip.html which can be accessed on the world-wide web using the “www” prefix). Pesticidal proteins also include toxin complex (TC) proteins, obtainable from organisms such as Xenorhabdus, Photorhabdus and Paenibacillus (see, U.S. Pat. Nos. 7,491,698 and 8,084,418). Some TC proteins have “stand alone” insecticidal activity and other TC proteins enhance the activity of the stand-alone toxins produced by the same given organism. The toxicity of a “stand-alone” TC protein (from Photorhabdus, Xenorhabdus or Paenibacillus, for example) can be enhanced by one or more TC protein “potentiators” derived from a source organism of a different genus. There are three main types of TC proteins. As referred to herein, Class A proteins (“Protein A”) are stand-alone toxins. Class B proteins (“Protein B”) and Class C proteins (“Protein C”) enhance the toxicity of Class A proteins. Examples of Class A proteins are TcbA, TcdA, XptA1 and XptA2. Examples of Class B proteins are TcaC, TcdB, XptB1Xb and XptC1Wi. Examples of Class C proteins are TccC, XptC1Xb and XptB1Wi. Pesticidal proteins also include spider, snake and scorpion venom proteins. Examples of spider venom peptides include but are not limited to lycotoxin-1 peptides and mutants thereof (U.S. Pat. No. 8,334,366).

The examples below describe some representative protocols and techniques for simulating plant insect feeding conditions and/or evaluating plants under such conditions.

1. Progeny of a transformed plant which is hemizygous with respect to a recombinant DNA construct, such that the progeny are segregating into plants either comprising or not comprising the recombinant DNA construct: the progeny comprising the recombinant DNA construct would be typically measured relative to the progeny not comprising the recombinant DNA construct (i.e., the progeny not comprising the recombinant DNA construct is the control or reference plant).

2. Introgression of a recombinant DNA construct into an inbred line, such as in maize, or into a variety, such as in soybean: the introgressed line would typically be measured relative to the parent inbred or variety line (i.e., the parent inbred or variety line is the control or reference plant).

3. Two hybrid lines, wherein the first hybrid line is produced from two parent inbred lines, and the second hybrid line is produced from the same two parent inbred lines except that one of the parent inbred lines contains a recombinant DNA construct: the second hybrid line would typically be measured relative to the first hybrid line (i.e., the first hybrid line is the control or reference plant).

4. A plant comprising a recombinant DNA construct: the plant may be assessed or measured relative to a control plant not comprising the recombinant DNA construct but otherwise having a comparable genetic background to the plant (e.g., sharing at least 90%, 91%, 92%, 93%, 94%, 95%, 96%, 97%, 98%, 99%, or 100% sequence identity of nuclear genetic material compared to the plant comprising the recombinant DNA construct). There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s), and Simple Sequence Repeats (SSRs) which are also referred to as Microsatellites.

Furthermore, one of ordinary skill in the art would readily recognize that a suitable control or reference plant to be utilized when assessing or measuring an agronomic characteristic or phenotype of a transgenic plant would not include a plant that had been previously selected, via mutagenesis or transformation, for the desired agronomic characteristic or phenotype.

“Pest” includes but is not limited to, insects, fungi, bacteria, nematodes, mites, ticks and the like. Insect pests include insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, etc., particularly Lepidoptera and Coleoptera.

Those skilled in the art will recognize that not all compounds are equally effective against all pests. Compounds of the embodiments display activity against insect pests, which may include economically important agronomic, forest, greenhouse, nursery ornamentals, food and fiber, public and animal health, domestic and commercial structure, household and stored product pests.

Larvae of the order Lepidoptera include, but are not limited to, armyworms, cutworms, loopers and heliothines in the family Noctuidae including Spodoptera frugiperda J E Smith (fall armyworm); S. exigua Hübner (beet armyworm); S. litura Fabricius (tobacco cutworm, cluster caterpillar); Mamestra configurata Walker (bertha armyworm); M. brassicae Linnaeus (cabbage moth); Agrotis ipsilon Hufnagel (black cutworm); A. orthogonia Morrison (western cutworm); A. subterranea Fabricius (granulate cutworm); Alabama argillacea Hübner (cotton leaf worm); Trichoplusia ni Hübner (cabbage looper); Pseudoplusia includens Walker (soybean looper); Anticarsia gemmatalis Hübner (velvetbean caterpillar); Hypena scabra Fabricius (green cloverworm); Heliothis virescens Fabricius (tobacco budworm); Pseudaletia unipuncta Haworth (armyworm); Athetis mindara Barnes and Mcdunnough (rough skinned cutworm); Euxoa messoria Harris (darksided cutworm); Earias insulana Boisduval (spiny bollworm); E. vittella Fabricius (spotted bollworm); Helicoverpa armigera Hübner (American bollworm); H. zea Boddie (corn earworm or cotton bollworm); Melanchra picta Harris (zebra caterpillar); Egira (Xylomyges) curialis Grote (citrus cutworm); Mythimna separate (Oriental Armyworm); borers, casebearers, webworms, coneworms, grass moths from the family Crambidae including Ostrinia fumacalis (Asian Corn Borer) and Ostrinia nubilalis (European Corn Borer), and skeletonizers from the family Pyralidae Ostrinia nubilalis Hübner (European corn borer); Amyelois transitella Walker (naval orangeworm); Anagasta kuehniella Zeller (Mediterranean flour moth); Cadra cautella Walker (almond moth); Chilo suppressalis Walker (rice stem borer); C. partellus, (sorghum borer); Corcyra cephalonica Stainton (rice moth); Crambus caliginosellus Clemens (corn root webworm); C. teterrellus Zincken (bluegrass webworm); Cnaphalocrocis medinalis Guenée (rice leaf roller); Desmia funeralis Hübner (grape leaffolder); Diaphania hyalinata Linnaeus (melon worm); D. nitidalis Stoll (pickleworm); Diatraea grandiosella Dyar (southwestern corn borer), D. saccharalis Fabricius (surgarcane borer); Eoreuma loftini Dyar (Mexican rice borer); Ephestia elutella Hübner (tobacco (cacao) moth); Galleria mellonella Linnaeus (greater wax moth); Herpetogramma licarsisalis Walker (sod webworm); Homoeosoma electellum Hulst (sunflower moth); Elasmopalpus lignosellus Zeller (lesser cornstalk borer); Achroia grisella Fabricius (lesser wax moth); Loxostege sticticalis Linnaeus (beet webworm); Orthaga thyrisalis Walker (tea tree web moth); Maruca testulalis Geyer (bean pod borer); Plodia interpunctella Hübner (Indian meal moth); Scirpophaga incertulas Walker (yellow stem borer); Udea rubigalis Guenée (celery leaftier); and leafrollers, budworms, seed worms and fruit worms in the family Tortricidae Acleris gloverana Walsingham (Western blackheaded budworm); A. variana Fernald (Eastern blackheaded budworm); Archips argyrospila Walker (fruit tree leaf roller); A. rosana Linnaeus (European leaf roller); and other Archips species, Adoxophyes orana Fischer von Rosslerstamm (summer fruit tortrix moth); Cochylis hospes Walsingham (banded sunflower moth); Cydia latiferreana Walsingham (filbertworm); C. pomonella Linnaeus (coding moth); Platynota flavedana Clemens (variegated leafroller); P. stultana Walsingham (omnivorous leafroller); Lobesia botrana Denis & Schiffermüller (European grape vine moth); Spilonota ocellana Denis & Schiffermüller (eyespotted bud moth); Endopiza viteana Clemens (grape berry moth); Eupoecilia ambiguella Hübner (vine moth); Bonagota salubricola Meyrick (Brazilian apple leafroller); Grapholita molesta Busck (oriental fruit moth); Suleima helianthana Riley (sunflower bud moth); Argyrotaenia spp.; Choristoneura spp.

Selected other agronomic pests in the order Lepidoptera include, but are not limited to, Alsophila pometaria Harris (fall cankerworm); Anarsia lineatella Zeller (peach twig borer); Anisota senatoria J. E. Smith (orange striped oakworm); Antheraea pernyi Guérin-Méneville (Chinese Oak Tussah Moth); Bombyx mori Linnaeus (Silkworm); Bucculatrix thurberiella Busck (cotton leaf perforator); Colias eurytheme Boisduval (alfalfa caterpillar); Datana integerrima Grote & Robinson (walnut caterpillar); Dendrolimus sibiricus Tschetwerikov (Siberian silk moth), Ennomos subsignaria Hübner (elm spanworm); Erannis tiliaria Harris (linden looper); Euproctis chrysorrhoea Linnaeus (browntail moth); Harrisina americana Guérin-Méneville (grapeleaf skeletonizer); Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Manduca quinquemaculata Haworth (five spotted hawk moth, tomato hornworm); M. sexta Haworth (tomato hornworm, tobacco hornworm); Operophtera brumata Linnaeus (winter moth); Paleacrita vernata Peck (spring cankerworm); Papilio cresphontes Cramer (giant swallowtail orange dog); Phryganidia californica Packard (California oakworm); Phyllocnistis citrella Stainton (citrus leafminer); Phyllonorycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval and Leconte (Southern cabbageworm); Sabulodes aegrotata Guenée (omnivorous looper); Schizura concinna J. E. Smith (red humped caterpillar); Sitotroga cerealella Olivier (Angoumois grain moth); Thaumetopoea pityocampa Schiffermuller (pine processionary caterpillar); Tineola bisselliella Hummel (webbing clothesmoth); Tuta absoluta Meyrick (tomato leafminer); Yponomeuta padella Linnaeus (ermine moth); Heliothis subflexa Guenée; Malacosoma spp. and Orgyia spp.

Of interest are larvae and adults of the order Coleoptera including weevils from the families Anthribidae, Bruchidae and Curculionidae (including, but not limited to: Anthonomus grandis Boheman (boll weevil); Lissorhoptrus oryzophilus Kuschel (rice water weevil); Sitophilus granarius Linnaeus (granary weevil); S. oryzae Linnaeus (rice weevil); Hypera punctata Fabricius (clover leaf weevil); Cylindrocopturus adspersus LeConte (sunflower stem weevil); Smicronyx fulvus LeConte (red sunflower seed weevil); S. sordidus LeConte (gray sunflower seed weevil); Sphenophorus maidis Chittenden (maize billbug)); flea beetles, cucumber beetles, rootworms, leaf beetles, potato beetles and leafminers in the family Chrysomelidae (including, but not limited to: Leptinotarsa decemlineata Say (Colorado potato beetle); Diabrotica virgifera virgifera LeConte (western corn rootworm); D. barberi Smith and Lawrence (northern corn rootworm); D. undecimpunctata howardi Barber (southern corn rootworm); Chaetocnema pulicaria Melsheimer (corn flea beetle); Phyllotreta cruciferae Goeze (Crucifer flea beetle); Phyllotreta striolata (stripped flea beetle); Colaspis brunnea Fabricius (grape colaspis); Oulema melanopus Linnaeus (cereal leaf beetle); Zygogramma exclamationis Fabricius (sunflower beetle)); beetles from the family Coccinellidae (including, but not limited to: Epilachna varivestis Mulsant (Mexican bean beetle)); chafers and other beetles from the family Scarabaeidae (including, but not limited to: Popillia japonica Newman (Japanese beetle); Cyclocephala borealis Arrow (northern masked chafer, white grub); C. immaculata Olivier (southern masked chafer, white grub); Rhizotrogus majalis Razoumowsky (European chafer); Phyllophaga crinita Burmeister (white grub); Ligyrus gibbosus De Geer (carrot beetle)); carpet beetles from the family Dermestidae; wireworms from the family Elateridae, Eleodes spp., Melanotus spp.; Conoderus spp.; Limonius spp.; Agriotes spp.; Ctenicera spp.; Aeolus spp.; bark beetles from the family Scolytidae and beetles from the family Tenebrionidae.

Adults and immatures of the order Diptera are of interest, including leafminers Agromyza parvicornis Loew (corn blotch leafminer); midges (including, but not limited to: Contarinia sorghicola Coquillett (sorghum midge); Mayetiola destructor Say (Hessian fly); Sitodiplosis mosellana Géhin (wheat midge); Neolasioptera murtfeldtiana Felt, (sunflower seed midge)); fruit flies (Tephritidae), Oscinella frit Linnaeus (fruit flies); maggots (including, but not limited to: Delia platura Meigen (seedcorn maggot); D. coarctata Fallen (wheat bulb fly) and other Delia spp., Meromyza americana Fitch (wheat stem maggot); Musca domestica Linnaeus (house flies); Fannia canicularis Linnaeus, F femoralis Stein (lesser house flies); Stomoxys calcitrans Linnaeus (stable flies)); face flies, horn flies, blow flies, Chrysomya spp.; Phormia spp. and other muscoid fly pests, horse flies Tabanus spp.; bot flies Gastrophilus spp.; Oestrus spp.; cattle grubs Hypoderma spp.; deer flies Chrysops spp.; Melophagus ovinus Linnaeus (keds) and other Brachycera, mosquitoes Aedes spp.; Anopheles spp.; Culex spp.; black flies Prosimulium spp.; Simulium spp.; biting midges, sand flies, sciarids, and other Nematocera.

Included as insects of interest are adults and nymphs of the orders Hemiptera and Homoptera such as, but not limited to, adelgids from the family Adelgidae, plant bugs from the family Miridae, cicadas from the family Cicadidae, leafhoppers, Empoasca spp.; Cicadella viridis (Linnaeus) from the family Cicadellidae, planthoppers from the families Cixiidae, Flatidae, Fulgoroidea, Issidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Asterolecanidae, Coccidae, Dactylopiidae, Diaspididae, Eriococcidae, Ortheziidae, Phoenicococcidae and Margarodidae, lace bugs from the family Tingidae, stink bugs from the family Pentatomidae, cinch bugs, Blissus spp.; and other seed bugs from the family Lygaeidae, spittlebugs from the family Cercopidae squash bugs from the family Coreidae and red bugs and cotton stainers from the family Pyrrhocoridae.

Agronomically important members from the order Homoptera further include, but are not limited to: Acyrthisiphon pisum Harris (pea aphid); Aphis craccivora Koch (cowpea aphid); A. fabae Scopoli (black bean aphid); A. gossypii Glover (cotton aphid, melon aphid); A. maidiradicis Forbes (corn root aphid); A. pomi De Geer (apple aphid); A. spiraecola Patch (spirea aphid); Aulacorthum solani Kaltenbach (foxglove aphid); Chaetosiphon fragaefolii Cockerell (strawberry aphid); Diuraphis noxia Kurdjumov/Mordvilko (Russian wheat aphid); Dysaphis plantaginea Paaserini (rosy apple aphid); Eriosoma lanigerum Hausmann (woolly apple aphid); Brevicoryne brassicae Linnaeus (cabbage aphid); Hyalopterus pruni Geoffroy (mealy plum aphid); Lipaphis erysimi Kaltenbach (turnip aphid); Metopolophium dirrhodum Walker (cereal aphid); Macrosiphum euphorbiae Thomas (potato aphid); Myzus persicae Sulzer (peach-potato aphid, green peach aphid); Nasonovia ribisnigri Mosley (lettuce aphid); Pemphigus spp. (root aphids and gall aphids); Rhopalosiphum maidis Fitch (corn leaf aphid); R. padi Linnaeus (bird cherry-oat aphid); Schizaphis graminum Rondani (greenbug); Sipha flava Forbes (yellow sugarcane aphid); Sitobion avenae Fabricius (English grain aphid); Therioaphis maculata Buckton (spotted alfalfa aphid); Toxoptera aurantii Boyer de Fonscolombe (black citrus aphid) and T. citricida Kirkaldy (brown citrus aphid); Adelges spp. (adelgids); Phylloxera devastatrix Pergande (pecan phylloxera); Bemisia tabaci Gennadius (tobacco whitefly, sweetpotato whitefly); B. argentifolii Bellows & Perring (silverleaf whitefly); Dialeurodes citri Ashmead (citrus whitefly); Trialeurodes abutiloneus (bandedwinged whitefly) and T. vaporariorum Westwood (greenhouse whitefly); Empoasca fabae Harris (potato leafhopper); Laodelphax striatellus Fallen (smaller brown planthopper); Macrolestes quadrilineatus Forbes (aster leafhopper); Nephotettix cinticeps Uhler (green leafhopper); N. nigropictus St∪1 (rice leafhopper); Nilaparvata lugens St∪1 (brown planthopper); Peregrinus maidis Ashmead (corn planthopper); Sogatella furcifera Horvath (white-backed planthopper); Sogatodes orizicola Muir (rice delphacid); Typhlocyba pomaria McAtee (white apple leafhopper); Erythroneoura spp. (grape leafhoppers); Magicicada septendecim Linnaeus (periodical cicada); Icerya purchasi Maskell (cottony cushion scale); Quadraspidiotus perniciosus Comstock (San Jose scale); Planococcus citri Risso (citrus mealybug); Pseudococcus spp. (other mealybug complex); Cacopsylla pyricola Foerster (pear psylla); Trioza diospyri Ashmead (persimmon psylla).

Agronomically important species of interest from the order Hemiptera include, but are not limited to: Acrosternum hilare Say (green stink bug); Anasa tristis De Geer (squash bug); Blissus leucopterus leucopterus Say (chinch bug); Corythuca gossypii Fabricius (cotton lace bug); Cyrtopeltis modesta Distant (tomato bug); Dysdercus suturellus Herrich-Schïffer (cotton stainer); Euschistus servus Say (brown stink bug); E. variolarius Palisot de Beauvois (one-spotted stink bug); Graptostethus spp. (complex of seed bugs); Leptoglossus corculus Say (leaf-footed pine seed bug); Lygus lineolaris Palisot de Beauvois (tarnished plant bug); L. Hesperus Knight (Western tarnished plant bug); L. pratensis Linnaeus (common meadow bug); L. rugulipennis Poppius (European tarnished plant bug); Lygocoris pabulinus Linnaeus (common green capsid); Nezara viridula Linnaeus (southern green stink bug); Oebalus pugnax Fabricius (rice stink bug); Oncopeltus fasciatus Dallas (large milkweed bug); Pseudatomoscelis seriatus Reuter (cotton fleahopper).

Furthermore, embodiments may be effective against Hemiptera such, Calocoris norvegicus Gmelin (strawberry bug); Orthops campestris Linnaeus; Plesiocoris rugicollis Fallen (apple capsid); Cyrtopeltis modestus Distant (tomato bug); Cyrtopeltis notatus Distant (suckfly); Spanagonicus albofasciatus Reuter (whitemarked fleahopper); Diaphnocoris chlorionis Say (honeylocust plant bug); Labopidicola allii Knight (onion plant bug); Pseudatomoscelis seriatus Reuter (cotton fleahopper); Adelphocoris rapidus Say (rapid plant bug); Poecilocapsus lineatus Fabricius (four-lined plant bug); Nysius ericae Schilling (false chinch bug); Nysius raphanus Howard (false chinch bug); Nezara viridula Linnaeus (Southern green stink bug); Eurygaster spp.; Coreidae spp.; Pyrrhocoridae spp.; Tinidae spp.; Blostomatidae spp.; Reduviidae spp. and Cimicidae spp.

Also included are adults and larvae of the order Acari (mites) such as Aceria tosichella Keifer (wheat curl mite); Petrobia latens Willer (brown wheat mite); spider mites and red mites in the family Tetranychidae, Panonychus ulmi Koch (European red mite); Tetranychus urticae Koch (two spotted spider mite); (T. mcdanieli McGregor (McDaniel mite); T. cinnabarinus Boisduval (carmine spider mite); T. turkestani Ugarov & Nikolski (strawberry spider mite); flat mites in the family Tenuipalpidae, Brevipalpus lewisi McGregor (citrus flat mite); rust and bud mites in the family Eriophyidae and other foliar feeding mites and mites important in human and animal health, i.e., dust mites in the family Epidermoptidae, follicle mites in the family Demodicidae, grain mites in the family Glycyphagidae, ticks in the order Ixodidae. Ixodes scapularis Say (deer tick); I. holocyclus Neumann (Australian paralysis tick); Dermacentor variabilis Say (American dog tick); Amblyomma americanum Linnaeus (lone star tick) and scab and itch mites in the families' Psoroptidae, Pyemotidae and Sarcoptidae.

Insect pests of the order Thysanura are of interest, such as Lepisma saccharina Linnaeus (silverfish); Thermobia domestica Packard (firebrat).

Additional arthropod pests covered include: spiders in the order Araneae such as Loxosceles reclusa Gertsch and Mulaik (brown recluse spider) and the Latrodectus mactans Fabricius (black widow spider) and centipedes in the order Scutigeromorpha such as Scutigera coleoptrata Linnaeus (house centipede).

Insect pest of interest include the superfamily of stink bugs and other related insects including but not limited to species belonging to the family Pentatomidae (Nezara viridula, Halyomorpha halys, Piezodorus guildini, Euschistus servus, Acrosternum hilare, Euschistus heros, Euschistus tristigmus, Dichelops furcatus, Dichelops melacanthus, and Bagrada hilaris (Bagrada Bug)), the family Plataspidae (Megacopta cribraria—Bean plataspid) and the family Cydnidae (Scaptocoris castanea—Root stink bug) and Lepidoptera species including but not limited to: diamond-back moth, e.g., Helicoverpa zea Boddie; soybean looper, e.g., Pseudoplusia includens Walker and velvet bean caterpillar e.g., Anticarsia gemmatalis Hübner.

Nematodes include parasitic nematodes such as root-knot, cyst and lesion nematodes, including Heterodera spp., Meloidogyne spp. and Globodera spp.; particularly members of the cyst nematodes, including, but not limited to, Heterodera glycines (soybean cyst nematode); Heterodera schachtii (beet cyst nematode); Heterodera avenae (cereal cyst nematode) and Globodera rostochiensis and Globodera pailida (potato cyst nematodes). Lesion nematodes include Pratylenchus spp.

Methods for measuring pesticidal activity are well known in the art. See, for example, Czapla and Lang, (1990) J. Econ. Entomol. 83:2480-2485; Andrews, et al., (1988) Biochem. J. 252:199-206; Marrone, et al., (1985) J. of Economic Entomology 78:290-293 and U.S. Pat. No. 5,743,477, all of which are herein incorporated by reference in their entirety. Generally, the protein is mixed and used in feeding assays. See, for example Marrone, et aL, (1985) J. of Economic Entomology 78:290-293. Such assays can include contacting plants with one or more pests and determining the plant's ability to survive and/or cause the death of the pests.

As used herein, the term “pesticidal activity” is used to refer to activity of an organism or a substance (such as, for example, a protein), whether toxic or inhibitory, that can be measured by, but is not limited to, pest mortality, pest weight loss, pest repellency, pest growth stunting, and other behavioral and physical changes of a pest after feeding and exposure for an appropriate length of time. In this manner, pesticidal activity impacts at least one measurable parameter of pest fitness. Similarly, “insecticidal activity” may be used to refer to “pesticidal activity” when the pest is an insect pest. “Stunting” is intended to mean greater than 50% inhibition of growth as determined by weight. General procedures for monitoring insecticidal activity include addition of the experimental compound or organism to the diet source in an enclosed container. Assays for assessing insecticidal activity are well known in the art. See, e.g., U.S. Pat. Nos. 6,570,005 and 6,339,144; herein incorporated by reference in their entirety. The optimal developmental stage for testing for insecticidal activity is larvae or immature forms of an insect of interest. The insects may be reared in total darkness at about 20˜30° C. and about 30%˜70% relative humidity. Bioassays may be performed as described in Czapla and Lang (1990) J. Econ. Entomol. 83(6):2480-2485. Methods of rearing insect larvae and performing bioassays are well known to one of ordinary skill in the art.

Toxic and inhibitory effects of insecticidal proteins include, but are not limited to, stunting of larval growth, killing eggs or larvae, reducing either adult or juvenile feeding on transgenic plants relative to that observed on wild-type, and inducing avoidance behavior in an insect as it relates to feeding, nesting, or breeding as described herein, insect resistance can be conferred to an organism by introducing a nucleotide sequence encoding an insecticidal protein or applying an insecticidal substance, which includes, but is not limited to, an insecticidal protein, to an organism (e.g., a plant or plant part thereof). As used herein, “controlling a pest population” or “controls a pest” refers to any effect on a pest that results in limiting the damage that the pest causes. Controlling a pest includes, but is not limited to, killing the pest, inhibiting development of the pest, altering fertility or growth of the pest in such a manner that the pest provides less damage to the plant, decreasing the number of offspring produced, producing less fit pests, producing pests more susceptible to predator attack or deterring the pests from eating the plant.

Methods

Methods include but are not limited to methods for increasing tolerance in a plant to an insect pest, methods for evaluating insect resistance, methods for controlling an insect population, methods for killing an insect population, methods for controlling an insect population resistance to an insecticidal polypeptide, and methods for producing seed. The plant may be a monocotyledonous or dicotyledonous plant, for example, a rice, maize, Arabidopsis, soybean plant. The plant may also be sunflower, sorghum, canola, wheat, alfalfa, cotton, barley or millet. The seed may be a rice, maize, Arabidopsis or soybean seed, for example a maize hybrid seed or maize inbred seed.

Methods include but are not limited to the following:

A method for transforming a cell comprising transforming a cell with any of the isolated polynucleotides of the present disclosure. The cell transformed by this method is also included. In particular embodiments, the cell is eukaryotic cell, e.g., a yeast, insect or plant cell, or prokaryotic, e.g., a bacterium.

A method for producing a transgenic plant comprising transforming a plant cell with any of the isolated polynucleotides or recombinant DNA constructs of the present disclosure and regenerating a transgenic plant from the transformed plant cell. The disclosure is also directed to the transgenic plant produced by this method, and transgenic seed obtained from this transgenic plant.

A method for isolating a polypeptide of the disclosure from a cell or culture medium of the cell, wherein the cell comprises a recombinant DNA construct comprising a polynucleotide of the disclosure operably linked to at least one regulatory sequence, and wherein the transformed host cell is grown under conditions that are suitable for expression of the recombinant DNA construct.

A method of altering the level of expression of a polypeptide of the disclosure in a host cell comprising: (a) transforming a host cell with a recombinant DNA construct of the present disclosure; and (b) growing the transformed host cell under conditions that are suitable for expression of the recombinant DNA construct wherein expression of the recombinant DNA construct results in production of altered levels of the polypeptide of the disclosure in the transformed host cell.

A method of increasing tolerance in a plant to an insect pest comprising: (a) introducing into a regenerable plant cell a recombinant DNA construct comprising a polynucleotide operably linked to at least one regulatory sequence (for example, a promoter functional in a plant), wherein the polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO: 6 or 14; and (b) regenerating a transgenic plant from the regenerable plant cell after step (a), wherein the transgenic plant comprises in its genome the recombinant DNA construct and exhibits increased tolerance to an insect pest when compared to a control plant not comprising the recombinant DNA construct. The method may further comprise (c) obtaining a progeny plant derived from the transgenic plant, wherein said progeny plant comprises in its genome the recombinant DNA construct and exhibits increased tolerance to an insect pest when compared to a control plant not comprising the recombinant DNA construct.

A method of increasing tolerance in a plant to an insect pest, comprising: (a) introducing into a regenerable plant cell a DNA construct comprising at least one heterologous regulatory element operably linked to a nucleic acid sequence encoding a CRK6 or MFS5 polypeptide in the plant genome; and (b) regenerating a transgenic plant from the regenerable plant cell after step (a), wherein the transgenic plant comprises in its genome the DNA construct, has increased expression of the CRK6 or MFS5 polypeptide, and exhibits increased tolerance to an insect pest when compared to a control plant not comprising the DNA construct. The method may further comprise (c) obtaining a progeny plant derived from the transgenic plant, wherein said progeny plant comprises in its genome the DNA construct, has increased expression of the CRK6 or MFS5 polypeptide and exhibits increased tolerance to an insect pest compared to a control plant not comprising the DNA construct.

In some embodiments methods are provided for controlling an insect pest comprising over-expressing in a plant a CRK6 or MFS5 polypeptide. In some embodiments the method for controlling an insect pest comprises transforming a plant or plant cell with the DNA constructs of the present disclosure.

In some embodiments methods are provided for killing an insect pest comprising over expressing in a plant a CRK6 or MFS5 polypeptide. In some embodiments the method for killing an insect pest comprises transforming a plant or plant cell with the DNA constructs of the present disclosure.

A method of evaluating tolerance to an insect pest in a plant, comprising (a) introducing into a regenerable plant cell a recombinant DNA construct comprising a polynucleotide operably linked to at least one regulatory sequence (for example, a promoter functional in a plant), wherein the polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity when compared to SEQ ID NO: 6 or 14; (b) regenerating a transgenic plant from the regenerable plant cell after step (a), wherein the transgenic plant comprises in its genome the recombinant DNA construct; and (c) evaluating the transgenic plant for insect tolerance compared to a control plant not comprising the recombinant DNA construct. The method may further comprise (d) obtaining a progeny plant derived from the transgenic plant, wherein the progeny plant comprises in its genome the recombinant DNA construct; and (e) evaluating the progeny plant for insect tolerance compared to a control plant not comprising the recombinant DNA construct.

A method of producing seed comprising any of the preceding methods, and further comprising obtaining seeds from said progeny plant, wherein said seeds comprise in their genome said recombinant DNA construct.

In some embodiments the disclosure provides seeds that comprise in their genome the recombinant DNA construct of the disclosure.

Seed Treatment

To protect and to enhance yield production and trait technologies, seed treatment options can provide additional crop plan flexibility and cost effective control against insects, weeds and diseases. Seed material can be treated with one or more of the insecticidal proteins or polypeptides disclosed herein. For e.g., such seed treatments can be applied on seeds that contain a transgenic trait including transgenic corn, soy, brassica, cotton or rice. Combinations of one or more of the insecticidal proteins or polypeptides disclosed herein and other conventional seed treatments are contemplated. Seed material can be treated, typically surface treated, with a composition comprising combinations of chemical or biological herbicides, herbicide safeners, insecticides, fungicides, germination inhibitors and enhancers, nutrients, plant growth regulators and activators, bactericides, nematocides, avicides and/or molluscicides. These compounds are typically formulated together with further carriers, surfactants or application-promoting adjuvants customarily employed in the art of formulation. The coatings may be applied by impregnating propagation material with a liquid formulation or by coating with a combined wet or dry formulation. Examples of the various types of compounds that may be used as seed treatments are provided in The Pesticide Manual: A World Compendium, C. D. S. Tomlin Ed., and Published by the British Crop Production Council, which is hereby incorporated by reference.

Some seed treatments that may be used on crop seed include, but are not limited to, one or more of abscisic acid, acibenzolar-S-methyl, avermectin, amitrol, azaconazole, azospirillum, azadirachtin, azoxystrobin, Bacillus spp. (including one or more of cereus, firmus, megaterium, pumilis, sphaericus, subtilis and/or thuringiensis species), bradyrhizobium spp. (including one or more of betae, canariense, elkanii, iriomotense, japonicum, liaonigense, pachyrhizi and/or yuanmingense), captan, carboxin, chitosan, clothianidin, copper, cyazypyr, difenoconazole, etidiazole, fipronil, fludioxonil, fluoxastrobin, fluquinconazole, flurazole, fluxofenim, harpin protein, imazalil, imidacloprid, ipconazole, isoflavenoids, lipo-chitooligosaccharide, mancozeb, manganese, maneb, mefenoxam, metalaxyl, metconazole, myclobutanil, PCNB, penflufen, penicillium, penthiopyrad, permethrine, picoxystrobin, prothioconazole, pyraclostrobin, rynaxypyr, S-metolachlor, saponin, sedaxane, TCMTB, tebuconazole, thiabendazole, thiamethoxam, thiocarb, thiram, tolclofos-methyl, triadimenol, trichoderma, trifloxystrobin, triticonazole and/or zinc. PCNB seed coat refers to EPA Registration Number 00293500419, containing quintozen and terrazole. TCMTB refers to 2-(thiocyanomethylthio) benzothiazole.

Seed varieties and seeds with specific transgenic traits may be tested to determine which seed treatment options and application rates may complement such varieties and transgenic traits in order to enhance yield. For example, a variety with good yield potential but head smut susceptibility may benefit from the use of a seed treatment that provides protection against head smut, a variety with good yield potential but cyst nematode susceptibility may benefit from the use of a seed treatment that provides protection against cyst nematode, and so on. Likewise, a variety encompassing a transgenic trait conferring tolerance to an insect pest may benefit from the second mode of action conferred by the seed treatment, a variety encompassing a transgenic trait conferring herbicide resistance may benefit from a seed treatment with a safener that enhances the plants resistance to that herbicide, etc. Further, the good root establishment and early emergence that results from the proper use of a seed treatment may result in more efficient nitrogen use, a better ability to withstand drought and an overall increase in yield potential of a variety or varieties containing a certain trait when combined with a seed treatment.

In any of the preceding methods or any other embodiments of methods of the present disclosure, the step of determining an alteration of an agronomic characteristic in a transgenic plant, if applicable, may comprise determining whether the transgenic plant exhibits an alteration of at least one agronomic characteristic when compared, under varying environmental conditions, to a control plant not comprising the recombinant DNA construct.

In any of the preceding methods or any other embodiments of methods of the present disclosure, the step of determining an alteration of an agronomic characteristic in a progeny plant, if applicable, may comprise determining whether the progeny plant exhibits an alteration of at least one agronomic characteristic when compared, under varying environmental conditions, to a control plant not comprising the recombinant DNA construct.

In any of the preceding methods or any other embodiments of methods of the present disclosure, in said introducing step said regenerable plant cell may comprises a callus cell, an embryogenic callus cell, a gametic cell, a meristematic cell, or a cell of an immature embryo. The regenerable plant cells may derive from an inbred maize plant.

In any of the preceding methods or any other embodiments of methods of the present disclosure, said regenerating step may comprise: (i) culturing said transformed plant cells in a media comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other embodiments of methods of the present disclosure, alternatives exist for introducing into a regenerable plant cell a recombinant DNA construct comprising a polynucleotide operably linked to at least one regulatory sequence. For example, one may introduce into a regenerable plant cell a regulatory sequence (such as one or more enhancers, optionally as part of a transposable element), and then screen for an event in which the regulatory sequence is operably linked to an endogenous gene encoding a polypeptide of the instant disclosure.

The introduction of recombinant DNA constructs of the present disclosure into plants may be carried out by any suitable technique, including but not limited to direct DNA uptake, chemical treatment, electroporation, microinjection, cell fusion, infection, vector mediated DNA transfer, bombardment, or Agrobacterium mediated transformation. Techniques for plant transformation and regeneration have been described in International Patent Publication WO 2009/006276, the contents of which are herein incorporated by reference.

In addition, methods to modify or alter the host endogenous genomic DNA are available. This includes altering the host native DNA sequence or a pre-existing transgenic sequence including regulatory elements, coding and non-coding sequences. These methods are also useful in targeting nucleic acids to pre-engineered target recognition sequences in the genome. As an example, the genetically modified cell or plant described herein, is generated using “custom” engineered endonucleases such as meganucleases produced to modify plant genomes (e.g., WO 2009/114321; Gao et al. (2010) Plant Journal 1:176-187). Another site-directed engineering is through the use of zinc finger domain recognition coupled with the restriction properties of restriction enzyme (e.g., Urnov, et al. (2010) Nat Rev Genet. 11(9):636-46; Shukla, et al. (2009) Nature 459 (7245):437-41). A transcription activator-like (TAL) effector-DNA modifying enzyme (TALE or TALEN) is also used to engineer changes in plant genome. See e.g., US20110145940, Cermak et al., (2011) Nucleic Acids Res. 39(12) and Boch et al., (2009), Science 326 (5959): 1509-12. Site-specific modification of plant genomes can also be performed using the bacterial type II CRISPR (clustered regularly interspaced short palindromic repeats)/Cas (CRISPR-associated) system. See e.g., Belhaj et al., (2013), Plant Methods 9: 39; The CRISPR/Cas system allows targeted cleavage of genomic DNA guided by a customizable small noncoding RNA.

The development or regeneration of plants containing the foreign, exogenous isolated nucleic acid fragment that encodes a protein of interest is well known in the art. The regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

### Stacking of Traits in Transgenic Plant

Transgenic plants may comprise a stack of one or more insecticidal or insect tolerance polynucleotides disclosed herein with one or more additional polynucleotides resulting in the production or suppression of multiple polypeptide sequences. Transgenic plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising a gene disclosed herein with a subsequent gene and cotransformation of genes into a single plant cell. As used herein, the term “stacked” includes having the multiple traits present in the same plant (i.e., both traits are incorporated into the nuclear genome, one trait is incorporated into the nuclear genome and one trait is incorporated into the genome of a plastid or both traits are incorporated into the genome of a plastid). In one non-limiting example, “stacked traits” comprise a molecular stack where the sequences are physically adjacent to each other. A trait, as used herein, refers to the phenotype derived from a particular sequence or groups of sequences. Co-transformation of genes can be carried out using single transformation vectors comprising multiple genes or genes carried separately on multiple vectors. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO 1999/25821, WO 1999/25854, WO 1999/25840, WO 1999/25855 and WO 1999/25853, all of which are herein incorporated by reference.

### EXAMPLES

The present disclosure is further illustrated in the following examples, in which parts and percentages are by weight and degrees are Celsius, unless otherwise stated. It should be understood that these examples, while indicating embodiments of the disclosure, are given by way of illustration only. From the above discussion and these examples, one skilled in the art can ascertain the essential characteristics of this disclosure, and without departing from the spirit and scope thereof, can make various changes and modifications of the disclosure to adapt it to various usages and conditions. Furthermore, various modifications of the disclosure in addition to those shown and described herein will be apparent to those skilled in the art from the foregoing description. Such modifications are also intended to fall within the scope of the appended claims.

### Example 1

### Creation of a Rice Population with an Activation-Tagging Construct

A binary construct that contains four multimerized enhancers elements derived from the Cauliflower Mosaic Virus 35S (CaMV 35S) promoter was used, and the rice activation tagging population was developed from Zhonghua 11 (Oryza sativa L.) which was transformed by Agrobacteria-mediated transformation method as described by Lin and Zhang ((2005) Plant Cell Rep. 23:540-547). Zhonghua 11 was cultivated by the Institute of Crop Sciences, Chinese Academy of Agricultural Sciences. The first batch of seeds used in this research was provided by Beijing Weiming Kaituo Agriculture Biotech Co., Ltd. Calli induced from embryos was transformed with Agrobacteria with the vector. The transgenic lines generated were developed and the transgenic seeds were harvested to form the rice activation tagging population.

### Example 2

### Seedling Screen to Identify Lines with Enhanced Tolerance to Asian Corn Borer (Ostrinia furnacalis) Insect Under Laboratory Conditions

Asian corn borer (ACB) (Ostrinia furnacalis (Guenée)) is an important insect pest for maize in Asia. This insect is distributed from China to Australia and the Solomon Islands. In northern parts of its range, the moths have one or a few generations per year, but in the tropics, generations are continuous and overlapping. The caterpillars can cause severe yield losses in corn, both by damage to the kernels and by feeding on the tassels, leaves, and stalks. Survival and growth of the caterpillar is highest on the reproductive parts of the plant. Other economic plants attacked include bell pepper, ginger and sorghum. Recently, the Asian corn borer appears to have become an important pest of cotton. A number of wild grasses are also used as hosts (D. M. Nafusa & I. H. Schreinera. 2012. Review of the biology and control of the Asian corn borer, Ostrinia furnacalis (Lep: Pyralidae). Tropical Pest Management. 37: 41-56).

ACB insect was used to identify rice ATLs which can inhibit larva development. Asian corn borer populations were obtained from the Institute of Plant Protection of Chinese Academy of Agricultural Sciences. This population was reared for more than 10 generations at 25-27° C., 60-80% relative humidity, under photo-period of 16L: 8D. The larvae were fed with artificial diet (Zhou Darong, Ye Zhihua, Wang Zhenying, 1995), and the eggs were hatched in incubator at 27° C. The newly hatched larvae were used in assays.

The T₂ seeds which showed red color under green fluorescent light (transgenic seeds) were used for insect tolerance assays except as otherwise specifically noted. One hundred fifty seeds of each activation tagged line (ATL) were sterilized by 800 ppm carbendazol for 8 h at 32° C. and washed 3-5 times, then placed on a layer of wet gauze in petri dash (12×12 cm). The germinated seeds were cultured in distilled water at 28° C. for 10 days and the seedlings which were 8-10 cm in height were used to feed ACB larvae.

### Screening Method:

The 32-well plates (4×4×2 cm for each well) (Pitman, N.J. USA-609-582-2392) were used and one-third volume of 1% agar solution was filled in each well to keep humidity. The 32-well plate could be divided into 8 blocks with each block of 4 wells for one rice ATL seedlings. Twenty rice seedlings without seeds and roots were inserted into the agar, six ACB neonate larvae were inoculated into the well with a brush, then special lids (Pitman, N.J. USA-609-582-2392) were covered the well. The tissue cultured ZH11 (ZH11-TC) were used as control, and the control seedlings were randomly placed in the blocks. The plates were placed in a chamber with temperature at 27.5° C. and 60% relative humidity, and rotated 90 degree each day from the second day. The insect larvae development was measured visually 5 days later, and the tolerant values were calculated.

The three largest larvae in each well were selected, compared with the larvae in the well with ZH11-TC seedlings, and then a tolerant value was obtained according to Table 2. If the larvae in the control well developed to third instar, then the larval development was considered as normal and the tolerant value is 0; if the larvae developed to second instar, it was smaller compared to the normal developed larvae and the tolerant value is 1; and if the larvae developed to first instar, it is very smaller and the tolerant value is 2.

Larvae growth inhibitory rate was used as a parameter for ACB insect tolerance assay, which is the percentage of the inhibited number over the statistics number of larvae, wherein the inhibited number of larvae is the sum of the tolerant value of 12 test insects from four wells in one repeat and the statistics number of larvae is the sum of the number of all the observed insects and number of larvae at 1st instar. Then the raw data were analyzed by Chi-square, the lines with P<0.01 were considered as ACB tolerance positive lines.

TABLE 2
Scoring Scales for Asian corn borer and Oriental armyworm assays

| Tolerant value | Instars of larvae | Size of larvae |
|---|---|---|
| 0 | 3^{rd} instar | Normal |
| 1 | 2^{nd} instar | Smaller |
| 2 | 1^{st} instar | Severe smaller |

The ACB tolerant lines from the primary screens will be re-screened in two continued screens (2^{nd} and 3^{rd} round of screens) with two repeats to confirm the insect tolerance. The ATLs which passed the 3^{rd} screens were considered as ACB tolerant lines.

### Screening Results:

### 1) AH43610 Seedlings

After ACB neonate larvae inoculating seedlings for 5 days in the screens, the seedlings of ZH11-TC were significantly damaged by ACB insects, while AH43610 seedling were less damaged, and the insects fed with AH43610 was smaller than that fed with ZH11-TC control. As shown in Table 3, ten of the 12 observed larvae with AH43610 seedlings developed to 2^{nd} instar, whereas 11 of the 12 observed insects with ZH11-TC seedlings grew normally into 3^{rd} instar. The larvae growth inhibitory rate of AH43610 was 83.33%, which was significantly greater than that of ZH11-TC seedlings (8.33%). These results show that AH43610 seedlings inhibited the development of ACB larvae. In the second screen, the larvae growth inhibitory rates of AH43610 in two repeats were 41.67% and 66.67%, respectively, whereas the larvae growth inhibitory rates of ZH11-TC controls both were 0.00%. The larvae growth inhibitory rates of AH43610 were significantly greater than ZH11-TC. One repeat of AH43610 in the 3^{rd} screening displayed the same trend, and in the other repeat, AH43610 exhibited greater larvae growth inhibitory rate. These results consistently demonstrate that feeding ACB with AH43610 seedlings can prevent the ACB larvae from developing into adults.

TABLE 3
Asian corn borer assay of AH43610 seedlings under laboratory screening condition

| | | | | Number of | | | |
|---|---|---|---|---|---|---|---|
| | | Number of | Number of | total | Larvae growth | | |
| | Screening | larvae at 1^{st} | larvae at 2^{nd} | observed | inhibitory rate | P | |
| Line ID | round | instar | instar | larvae | (%) | value | P ≤ 0.01 |
| ZH11-TC | 1-1 | 0 | 1 | 12 | 8.33 | | |
| AH43610 | | 0 | 10 | 12 | 83.33 | 0.0002 | Y |
| ZH11-TC | 2-1 | 0 | 0 | 12 | 0.00 | | |
| AH43610 | | 0 | 5 | 12 | 41.67 | 0.0120 | |
| ZH11-TC | 2-2 | 0 | 0 | 12 | 0.00 | | |
| AH43610 | | 0 | 6 | 9 | 66.67 | 0.0008 | Y |
| ZH11-TC | 3-1 | 0 | 0 | 22 | 0.00 | | |
| AH43610 | | 0 | 5 | 18 | 27.78 | 0.0082 | Y |
| ZH11-TC | 3-2 | 0 | 6 | 24 | 25.00 | | |
| AH43610 | | 0 | 6 | 21 | 28.57 | 0.7869 | |

### 2) AH29691 Seedlings

After ACB neonate larvae inoculating seedlings for 5 days in the screens, the seedlings of ZH11-TC were significantly damaged by ACB insects, while AH29691 seedling were less damaged, and the insects fed with AH29691 was smaller than that fed with ZH11-TC control. Table 4 shows the three rounds screening results for AH29691 seedlings. In the first screening, six insects in AH29691 seedlings' wells developed into 2^{nd} instar, while all observed 12 insects fed with ZH11-TC seedlings normally grew into 3^{rd} instar. The larvae growth inhibitory rate of AH29691 (50%) was significantly greater than that of ZH11-TC seedlings (0.00%). These results indicated that AH29691 seedlings inhibited the development of ACB larvae. Therefore, it was further screened. In the second screening, the larvae growth inhibitory rates of AH29691 in two repeats were 50% and 66.67%, respectively, which were significantly greater than that of their corresponding ZH11-TC controls. The larvae growth inhibitory rates of AH29691 seedlings were also significantly greater than that of their corresponding ZH11-TC controls in two repeats of 3^{rd} round screening, respectively. These results clearly and consistently demonstrate that AH29691 seedling can inhibit the development of ACB insect and AH29691 was an ACB tolerant line.

TABLE 4
Asian corn borer assay of AH29691 seedlings under laboratory screening condition

| | | | Number | | Larvae | | |
|---|---|---|---|---|---|---|---|
| | | Number of | of larvae | Number of | growth | | |
| | Screening | lavae at | at 2^{nd} | total observed | inhibitory | | |
| Line ID | round | 1^{st} instar | instar | larvae | rate (%) | P value | P ≤ 0.01 |
| ZH11-TC | 1-1 | 0 | 0 | 12 | 0.00 | | |
| AH29691 | | 0 | 6 | 12 | 50.00 | 0.0047 | Y |
| ZH11-TC | | 0 | 0 | 12 | 0.00 | | |
| AH29691 | 2-1 | 0 | 6 | 12 | 50.00 | 0.0047 | Y |
| AH29691 | 2-2 | 0 | 6 | 9 | 66.67 | 0.0008 | Y |
| ZH11-TC | | 0 | 0 | 12 | 0.00 | | |
| AH29691 | 3-1 | 0 | 5 | 12 | 41.67 | 0.0120 | |
| AH29691 | 3-2 | 0 | 10 | 12 | 83.33 | 0.0000 | Y |

### Example 3

### Cross-Validation of ACB Tolerance ATLs with Oriental Armyworm (Mythimna separata) Under Laboratory Conditions

Oriental armyworm (OAW) was used in cross-validations of insecticidal activity. OAW belongs to Lepidoptera Noctuidae, and is a polyphagous insect pest. The eggs of OAW were obtained from the Institute of Plant Protection of Chinese Academy of Agricultural Sciences and hatched in an incubator at 27° C. The neonate larvae were used in this cross validation assay.

Rice ATL plants were cultured as described in Example 2, and the experiments design was similar as to ACB insect assay described in Example 2. Five days later, all the survived larvae were visually measured and given tolerant values according to Table 2.

Larvae growth inhibitory rate was used as a parameter for this insect tolerance assay, which is the percentage of the inhibited number over the statistics number of larvae, wherein the inhibited number is the sum of the tolerance value of all observed test insects from four wells in one repeat and the statistics number of larvae is the sum of the number of all the observed insects and number of larvae at 1st instar.

The raw data were analyzed by Chi-square, the lines with P<0.01 were considered as OAW tolerant positive lines.

### Screening Results:

Table 5 shows the OAW screening results of AH43610 and AH29691. No larva of all observed 22 larvae in four AH43610 wells developed to 3^{rd} instar, 15 larvae developed to 2^{nd} instar, and seven larvae developed to 1st instar; while nine larvae in the ZH11-TC control wells grew to 3^{rd} instar, 34 larvae grew to 2^{nd} instar and two larvae grew to 1st instar. The larvae growth inhibitory rate of AH43610 seedlings was 100%, which was significantly greater than that of ZH11-TC control (80.85%). One larva of all observed 17 larvae in four AH29691 wells developed to 1st instar, and ten larvae developed to 2^{nd} instar; while one larva grew to 1st instar and 20 larvae grew to 2^{nd} instar in the ZH11-TC control wells. The larvae growth inhibitory rate of AH29691 seedlings was slightly greater than that of ZH11-TC control. These results demonstrate that AH43610 seedlings inhibit the growth of OAW larvae, and AH29691 seedlings showed a small degree of enhanced resistance against OAW larave.

TABLE 5
Oriental armyworm assay of AH43610 and AH29691 seedlings under laboratory
screening condition

| | | | Number of | | | |
|---|---|---|---|---|---|---|
| | Number of | Number of | total | Larvae growth | | |
| | larvae at 1^{st} | larvae at 2^{nd} | observed | inhibitory rate | | |
| Line ID | instar | instar | larvae | (%) | P value | P ≤ 0.01 |
| ZH11-TC | 2 | 34 | 45 | 80.85 | | |
| AH43610 | 7 | 15 | 22 | 100.00 | 0.0121 | |
| ZH11-TC | 1 | 20 | 34 | 62.86 | | |
| AH29691 | 1 | 10 | 17 | 66.67 | 0.7842 | |

### Example 4

### Cross-Validation of ACB Tolerance Positive ATLs with Rice Stem Bore (Chilo suppressalis) Under Laboratory Screening Conditions

Rice stem borer (RCB) belongs to Lepidoptera Pyralidae and it is a very important rice pest. They infest plants from the seedling stage to maturity. Although worldwide in distribution, rice stem borers are particularly destructive in Asia, the Middle East, and the Mediterranean regions.

The eggs of RSB were obtained from the Institute of Plant Protection of Chinese Academy of Agricultural Sciences and hatched in an incubator at 27° C. The neonate larvae were used in this cross validation assay.

ATLs seedlings were cultured in greenhouse. Two types of lamps were provided as light source, i.e. sodium lamp and metal halide lamp, with the ratio of 1:1. Lamps provide the 16 h/8 h period of day/night, and were placed approximately 1.5 m above the seedbed. The light intensity 30 cm above the seedbed is measured as 10,000-20,000 lx in sunny day, while 6,000-10,000 lx in cloudy day, the relative humidity ranges from 30% to 90%, and the temperature ranges from 20 to 35° C. The tillered seedlings cultured with modified IRRI nutrient solution for 40-d were used in this assay.

### Screening Method:

Two main stems of ATLs or ZH11-TC rice plants cultured for 40-d were cut into 7-8 cm, and inserted into agar in an 100 mL triangular flask, and then 10 RSB neonate larvae were inoculated on the top of main stems with a brush in each triangular flask. The triangular flasks were placed in chamber with temperature at 27.5° C. and 70% relative humidity. The ZH11-TC main stems were used as control, and six repeats were designed in the experiments.

Mortality rate and larvae growth inhibitory rate were measured 7 day after inoculation. The mortality rate is the percentage of number of died larvae over the number of inoculated larvae, and the larvae growth inhibitory rate is the percentage of the sum of number of died larvae, number of larvae at 1^{st} instar and number of larvae at 2^{nd} instar over the number of inoculated larvae.

The raw data were analyzed by Chi-square, the lines with P<0.01 are considered as RSB tolerance positive lines.

### Screening Results:

### 1) AH43610 Stems

Of all the 30 RSB larvae fed with the AH43610 stems, 14 larvae died, five larvae grew into 1^{st} instar, and six larvae grew into 2^{nd} instar; while six larvae fed with ZH11-TC seedlings died, 15 larvae grew to 2^{nd} instar, and six larvae grew to 1^{st} instar; two larvae fed with AH43610 segregated non-fluorescent (AH43610-N) controls died, four larvae grew into 2^{nd} instar, and five larvae grew into 3^{rd} instar. The mortality rate and larvae growth inhibitory rate of AH43610 main stems were 46.67% and 83.33%, respectively. The mortality rate and larvae growth inhibitory rate of ZH11-TC controls were 15% and 60%, respectively. The mortality rate and larvae growth inhibitory rate of AH43610-N controls were 6.67% and 36.67%, respectively. These results clearly show that AH43610 can significantly inhibit the growth and development of RSB larvae.

### 2) AH29691 Stems

For AH29691 stems fed RSB larvae, 17 larvae died, two larvae developed to 1^{st} instar and seven larvae developed to 2^{nd} instar; whereas eight larvae fed with ZH11-TC controls died, and five larvae developed to 2^{nd} instar. The mortality rate and larvae growth inhibitory rate of AH29691 main stems were greater than that of ZH11-TC main stems, indicating that AH29691 seedlings can inhibit the growth of RSB larvae.

TABLE 6
Rice stem borer assay of AH43610 and AH29691seedlings under laboratory
screening condition

| | Number | Number | Number | Number | | | | |
|---|---|---|---|---|---|---|---|---|
| | of dead | of 1^{st} | of 2^{nd} | of total | Mortality | | Inhibited | |
| Line ID | larvae | instar | instar | larvae | rate (%) | P value | rate (%) | P value |
| ZH11-TC | 6 | 3 | 15 | 40 | 15.00 | | 60.00 | |
| AH43610 | 14 | 5 | 6 | 30 | 46.67 | 0.1268 | 83.33 | 0.2108 |
| AH43610-N | 2 | 4 | 5 | 30 | 6.67 | | 36.67 | |
| AH43610 | 14 | 5 | 6 | 30 | 46.67 | 0.0437 | 83.33 | 0.1347 |
| ZH11-TC | 8 | 0 | 5 | 60 | 13.33 | | 21.67 | |
| AH29691 | 17 | 2 | 7 | 60 | 28.33 | 0.0276 | 43.33 | 0.0837 |

AH43610 and AH29691 seedlings showed significant inhibitory impact on the growth and development of ACB, OAW and RSB insects, indicating the potential broad spectrum of insecticidal activities.

In light of these results, the gene(s) which contributed to the enhanced insect tolerance of Line AH43610 and AH29691 were isolated.

### Example 5

### Identification of Activation-Tagged Genes

Genes flanking the T-DNA insertion locus in the insect tolerant line AH43610 and AH29691 were identified using one, or both, of the following two standard procedures: (1) Plasmid Rescue (Friedrich J. Behringer and June I. Medford. (1992), Plant Molecular Biology Reporter Vol. 10, 2:190-198); and (2) Inverse PCR (M. J. McPherson and Philip Quirke. (1991), PCR: a practical approach, 137-146). For lines with complex multimerized T-DNA inserts, plasmid rescue and inverse PCR may both prove insufficient to identify candidate genes. In these cases, other procedures, including TAIL PCR (Liu et al. (1995), Plant J. 8:457-463) can be employed.

A successful sequencing result is one where a single DNA fragment contains a T-DNA border sequence and flanking genomic sequence. Once a tag of genomic sequence flanking a T-DNA insert is obtained, candidate genes are identified by alignment to publicly available rice genome sequence. Specifically, the annotated gene nearest the 35S enhancer elements/T-DNA RB are candidates for genes that are activated.

To verify that an identified gene is truly near a T-DNA and to rule out the possibility that the DNA fragment is a chimeric cloning artifact, a diagnostic PCR on genomic DNA is done with one oligo in the T-DNA and one oligo specific for the local genomic DNA. Genomic DNA samples that give a PCR product are interpreted as representing a T-DNA insertion. This analysis also verifies a situation in which more than one insertion event occurs in the same line, e.g., if multiple differing genomic fragments are identified in Plasmid Rescue and/or Inverse-PCR analyses.

Genomic DNA was isolated from leaf tissues of the AH43610 line and AH29691 line using CTAB method (Murray, M. G. and W. F. Thompson. (1980) Nucleic Acids Res. 8: 4321-4326).

The flanking sequences of T-DNA insertion locus were obtained by molecular technology.

The tandem T-DNAs were inserted between 9419566-9419587 bp in chromosome 3 of AH43610 (MSU7.0 http://rice.plantbiology.msu.edu/index.shtml). The nucleotide sequences of RB and LB flanking sequence of T-DNA in AH43610 were shown as SEQ ID NO: 1 and 2.

The T-DNA was inserted at 21101049 bp in chromosome 9 of AH29691 (MSU7.0 http://rice.plantbiology.msu.edu/index.shtml). The nucleotide sequences of RB flanking sequence of T-DNA in AH29691 were shown as SEQ ID NO: 11.

OsCRK6 gene is near the T-DNA insertion site in AH43610 line, and OsMFS5 gene is near the T-DNA insertion site in AH29691 line. These genes were cloned and validated as to its functions in insect tolerance and other agronomic trait improvement.

### Example 6

### Cloning and Over-Expression Vector Construction for Insect Tolerance Genes

Based on the sequence information of gene ID (LOC_Os03g16960.1 and LOC_Os09g36600.1), primers were designed for cloning rice insect tolerance genes. The primers and the expected-lengths of the amplified genes are shown in Table 7.

OsCRK6 cDNA was cloned from pooled cDNA from leaf, stem and root tissues of Chaoyou 1 plant and OsMFS5 cDNA was cloned from pooled cDNA from leaf, stem and root tissues of Zhonghua 11 plant. The PCR reaction mixtures and PCR procedures are shown in Table 8 and Table 9.

TABLE 7
Primers for cloning insect tolerance genes

| | | | | Length of |
|---|---|---|---|---|
| | | | | amplified |
| | | SEQ ID | | fragment |
| Primer | Sequence | NO: | Gene name | (bp) |
| gc-4363 | 5′-GAAACACACAGCATTGAGACTG-3′ | 7 | OsCRK6 | 867 |
| gc-4364 | 5′-CACCATGTATGTGTAGGTAGTTC-3′ | 8 | | |
| gc-9173 | 5′-CAACAGCAACCACTCCGACGAAC-3′ | 15 | OsMFS5 | 1746 |
| gc-9174 | 5′-GATGCAATTGCGCGAATCTGTACC-3′ | 16 | | |

TABLE 8
PCR reaction mixture

| | |
|---|---|
| Reaction mix | 50 μL |
| Template | 1 μL |
| TOYOBO KOD-FX (1.0 U/μL) | 1 μL |
| 2x PCR buffer for KOD-FX | 25 μL |
| 2 mM dNTPs (0.4 mM each) | 10 μL |
| Primer-F/R (10 μM) | 2 μL each |
| ddH₂O | 9 μL |

TABLE 9
PCR cycle conditions for cloning insect tolerance gene

| | | | |
|---|---|---|---|
| 94° C. | 3 min | | |
| 98° C. | 10 s | | |
| 58° C. | 30 s | {close oversize brace} | ×30 |
| 68° C. | (1 Kb/min) min | | |
| 68° C. | 5 min | | |

The PCR amplified products were extracted after the agarose gel electrophoresis using a column kit and then ligated with TA cloning vectors. The sequence and orientation in the construct was confirmed by sequencing. The genes were cloned into plant binary construct DP0158 (pCAMBIA1300-DsRed) (SEQ ID NO: 3). The cloned nucleotide sequence in construct of DP0482 and coding sequence of OsCRK6 are provided as SEQ ID NO: 4 and 5, the encoded amino acid sequence of OsCRK6 is SEQ ID NO: 6. The cloned nucleotide sequence in construct of DP1191 and coding sequence of OsMFS5 are provided as SEQ ID NO: 12 and 13, the encoded amino acid sequence of OsMFS5 is SEQ ID NO: 14.

### Example 7

### Transformation to Get the Transgenic Rice Lines

The over-expression vectors and empty vectors (DP0158) were transformed into Zhonghua 11 (Oryza sativa L.) by Agrobacteria-mediated method as described by Lin and Zhang ((2005) Plant Cell Rep. 23:540-547). The transgenic seedlings (T₀) generated in transformation laboratory were transplanted in the field to get T₁ seeds. The T₁ and T₂ seeds were stored at cold room (4° C.). The over-expression vectors contain DsRED and HYG genes. T₁ and T₂ seeds which showed red color under green fluorescent light were transgenic seeds and were used in the following insect tolerant assays.

Gene Expression Analysis in Transgenic Rice Plants:

Gene expression levels in the transgenic rice plants are analyzed by a standard real-time RT-PCR procedure, such as the QuantiTect® Reverse Transcription Kit from Qiagen® and Real-Time RT-PCR (SYBR^{R}Premix Ex Taq™, TaKaRa). EF1a gene is used as an internal control to show that the amplification and loading of samples from the transgenic rice and control plant are similar. The expression level is normalized based on the EF1α mRNA levels.

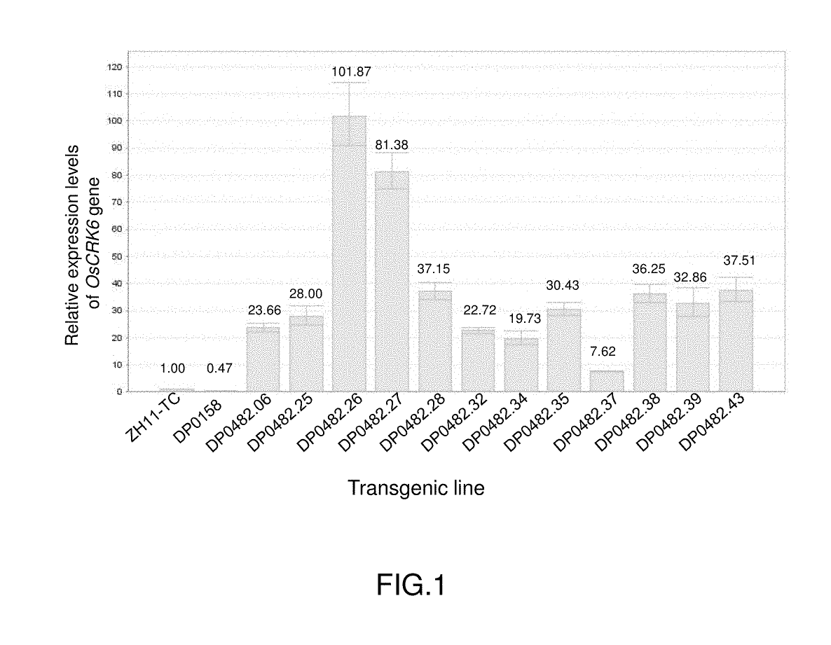

OsCRK6 gene expression levels in the DP0482 rice plants were detected using the following primers. As shown in FIG. 1, the expression level in ZH11-TC rice is set at 1.00, the gene expression level in DP0158 rice is similar to that of ZH11-TC, and OsCRK6 over-expressed in all the twelve lines.

DP0482-F1:
(SEQ ID NO: 9)
5′-GCCACTACCGACATGACAAAG-3′
DP0482-R1:
(SEQ ID NO: 10)
5′-GCATGCACATCACCATGTATG-3′

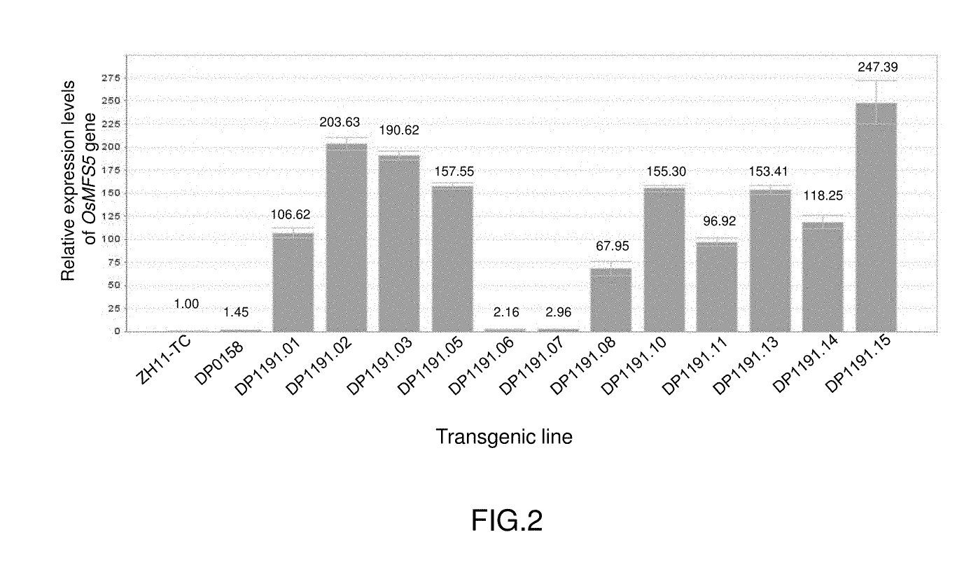

OsMFS5 gene expression levels in the DP1191 rice plants were detected using the following primers. As shown in FIG. 2, the expression level in ZH11-TC rice is set at 1.00, the gene expression level in DP0158 rice is similar to that of ZH11-TC, and OsMFS5 over-expressed in all the twelve lines.

DP1191-F1:
(SEQ ID NO: 17)
5′-GTTCGGTTTGGATGTCTTGC-3′
DP1191-R1:
(SEQ ID NO: 18)
5′-CTCTGCCTCTTGCTCTCATG-3′

### Example 8

### ACB Assay of OsCRK6 Transgenic Rice Plants Under Laboratory Conditions

In order to investigate whether OsCRK6 transgenic rice can recapitulate the insect tolerance trait of AH43610 line, the OsCRK6 transgenic rice was first tested against ACB insect. The ACB insect was reared as described in Example 2.

T₂ plants generated with the construct were tested in the assays for three times with four repeats. The seedlings of ZH11-TC and DP0158 were used as controls. Twelve lines transgenic rice were tested and 450 seeds of each line were water cultured for 10 days as described in Example 2. This recapitulation assay used randomized block design. Seedlings of each line were inserted in two wells of the 32-well-plate, and ZH11-TC and DP0158 seedlings were inserted in four different wells in the same plate.

Larvae growth inhibitory rate was used as a parameter for ACB insect tolerance assay, which is the percentage of the inhibited larvae number over the statistics number of larvae, wherein the inhibited larvae number is the sum of the tolerance value of test insects from eight wells and the statistics number of larvae is the sum of the number of all the observed insects and number of larvae at 1^{st} instar.

Randomized block design was used, and 12 transgenic lines from a construct were tested in one experimental unit to evaluate the gene function by SAS PROC GLIMMIX considering construct, line and environment effects. If the larvae growth inhibitory rates of the transgenic rice plants at both construct and line levels were significantly greater than controls (P<0.05), the gene was considered having ACB tolerant function.

### ACB Screening Results:

### 1) Results of the First Validation Experiment

After ACB neonate larvae inoculating seedlings for 5 days in the assays, the seedlings of ZH11-TC and DP0158 were significantly damaged by ACB insects, while the OsCRK6 transgenic seedlings were less damaged, and the insects fed with the OsCRK6 transgenic seedlings was smaller than that fed with ZH11-TC and DP0158 controls.

Twelve OsCRK6 transgenic lines were placed on one plate, and repeated for four times. A total of 576 ACB neonate larvae were inoculated on OsCRK6 transgenic rice seedlings. Five days after inoculation, 414 larvae were found, 14 larvae developed into 1^{st} instar, and 171 larvae developed to 2^{nd} instar. Only two larvae of all the observed 69 larvae in ZH11-TC seedlings' wells developed to 1^{st} instar and 23 larvae developed to 2^{nd} instar. Similar results were obtained with DP0158 seedlings, three larvae of all observed 79 larvae inoculated on the DP0158 seedling developed to 1^{st} instar, and 17 larvae developed to 2^{nd} instar. The average larvae growth inhibitory rates of OsCRK6 transgenic rice, ZH11-TC and DP0158 were 46.50%, 38.03% and 28.05%, respectively. The average larvae growth inhibitory rate of OsCRK6 transgenic rice was greater than that of ZH11-TC (P value=0.1617) and significantly greater than that of DP0158 (P value=0.0032) control. These results show that over-expression of OsCRK6 in rice significantly increased ACB insect tolerance of transgenic rice at construct level.

Further analysis at transgenic line level is displayed in Table 10. Ten transgenic lines exhibited greater larvae growth inhibitory rates than both ZH11-TC and DP0158 controls. Six lines exhibited significantly greater larvae growth inhibitory rates than DP0158 seedlings. These results further indicate OsCRK6 plays a role in increasing ACB insect tolerance in rice compared to controls at line level.

TABLE 10
Asian corn borer assay of OsCRK6 transgenic rice under laboratory screening
condition at line level (1^{st} experiment)

| | | | Number of | | | | | |
|---|---|---|---|---|---|---|---|---|
| | Number of | Number of | total | Larvae growth | CK = ZH11-TC | | CK = DP0158 | |
| | larvae at 1^{st} | larvae at | observed | inhibitory rate | P | | P | |
| Line ID | instar | 2^{nd} instar | larvae | (%) | value | P ≤ 0.05 | value | P ≤ 0.05 |
| ZH11-TC | 2 | 23 | 69 | 38.03 | | | | |
| DP0158 | 3 | 17 | 79 | 28.05 | | | | |
| DP0482.06 | 2 | 20 | 43 | 53.33 | 0.1128 | | 0.0080 | Y |
| DP0482.25 | 2 | 16 | 39 | 48.78 | 0.2661 | | 0.0290 | Y |
| DP0482.26 | 2 | 15 | 42 | 43.18 | 0.5893 | | 0.0964 | |
| DP0482.27 | 1 | 12 | 33 | 41.18 | 0.8068 | | 0.2013 | |
| DP0482.28 | 1 | 16 | 36 | 48.65 | 0.2980 | | 0.0370 | Y |
| DP0482.32 | 0 | 16 | 45 | 35.56 | 0.7943 | | 0.3841 | |
| DP0482.34 | 3 | 10 | 24 | 59.26 | 0.0749 | | 0.0077 | Y |
| DP0482.35 | 2 | 10 | 36 | 36.84 | 0.8897 | | 0.3489 | |
| DP0482.37 | 0 | 14 | 36 | 38.89 | 0.9414 | | 0.2569 | |
| DP0482.38 | 0 | 15 | 24 | 62.50 | 0.0528 | | 0.0057 | Y |
| DP0482.39 | 0 | 14 | 29 | 48.28 | 0.3865 | | 0.0667 | |
| DP0482.43 | 1 | 13 | 27 | 53.57 | 0.1779 | | 0.0226 | Y |

### 2) Results of the Second Validation Experiment

The same twelve OsCRK6 transgenic lines were tested in this second experiment, and the DP0158 seedlings were used as controls. Five days after inoculation, 504 larvae were found, six larvae developed into 1^{st} instar, and 223 larvae developed to 2^{nd} instar. Only two larvae of all the observed 88 larvae in DP0158 seedlings' wells developed to 1^{st} instar and 21 larvae developed to 2^{nd} instar. The average larvae growth inhibitory rates of OsCRK6 transgenic rice and DP0158 were 46.08% and 27.78%, respectively. The average larvae growth inhibitory rate of OsCRK6 transgenic rice was significantly greater than that of DP0158 (P value=0.0026) control. These results show that over-expression of OsCRK6 in rice increased ACB insect tolerance of transgenic rice at construct level.

Further analysis at transgenic line level is displayed in Table 11. Eleven transgenic lines exhibited greater larvae growth inhibitory rates than DP0158 seedlings, and nine lines exhibited the significantly greater larvae growth inhibitory rates. The larvae growth inhibitory rate of line DP0482.38 is 65%, is greatest. The result was same to that in the first validation experiment. These results further indicate OsCRK6 plays a role in increasing ACB insect tolerance in rice compared to controls at line level.

TABLE 11
Asian corn borer assay of OsCRK6 transgenic rice under laboratory screen
condition at line level (2^{nd} experiment)

| | Number | Number | Number | | | |
|---|---|---|---|---|---|---|
| | of larvae | of larvae | of total | Larvae growth | | |
| | at 1^{st} | at 2^{nd} | observed | inhibitory | | |
| Line ID | instar | instar | larvae | rate (%) | P value | P ≤ 0.05 |
| DP0158 | 2 | 21 | 88 | 27.78 | | |
| DP0482.06 | 0 | 20 | 39 | 51.28 | 0.0145 | Y |
| DP0482.25 | 2 | 18 | 42 | 50.00 | 0.0159 | Y |
| DP0482.26 | 0 | 9 | 38 | 23.68 | 0.6889 | |
| DP0482.27 | 0 | 19 | 41 | 46.34 | 0.0295 | Y |
| DP0482.28 | 0 | 12 | 42 | 28.57 | 0.8475 | |
| DP0482.32 | 0 | 21 | 41 | 51.22 | 0.0111 | Y |
| DP0482.34 | 1 | 21 | 46 | 48.94 | 0.0156 | Y |
| DP0482.35 | 0 | 23 | 46 | 50.00 | 0.0110 | Y |
| DP0482.37 | 0 | 16 | 45 | 35.56 | 0.3744 | |
| DP0482.38 | 1 | 24 | 39 | 65.00 | 0.0005 | Y |
| DP0482.39 | 0 | 24 | 43 | 55.81 | 0.0024 | Y |
| DP0482.43 | 2 | 16 | 42 | 45.45 | 0.0409 | Y |

### Example 9

OAW Assay of OsCRK6 Transgenic Rice Plants Under Laboratory Conditions

OAW assay of OsCRK6 transgenic rice were performed as described in Example 3. Larvae growth inhibitory rate was used as a parameter for this insect tolerance assay, which is the percentage of the inhibited number over the statistics number of larvae, wherein the inhibited number is the sum of the tolerance value of all observed test insects from eight wells and the statistics number of larvae is the sum of the number of all the observed insects and number of larvae at 1^{st} instar.

### OAW Screening Results:

Twelve transgenic lines which were tested in the ACB assay were used in this assay. These rice lines were placed in one 32-well plate with four repeats. Five days after larvae inoculation, three larvae of 488 larvae found in the OsCRK6 transgenic rice well developed to 1^{st} instar, and 113 larvae developed to 2^{nd} instar. The OAW larvae growth inhibitory rate was 24.24%. While, one of the 90 larvae in the ZH11-TC wells developed to 1^{st} instar, and 20 larvae developed to 2^{nd} instar. The larvae growth inhibitory rate of ZH11-TC seedlings was 24.18%. One of 90 larvae in the DP0158 seedling well developed to 1^{st} instar, and 15 larvae developed to 2^{nd} instar. The larvae growth inhibitory rate of DP0158 seedlings was 18.68%. The OAW larvae growth inhibitory rate of OsCRK6 transgenic rice was greater than ZH11-TC and DP0158 controls, but the differences did not reach significant.

TABLE 12
Armyworm assay of OsCRK6 transgenic rice under laboratory screen condition at
construct level (1^{st} experiment)

| | Number | Number | Number | Larvae | | | | |
|---|---|---|---|---|---|---|---|---|
| | of larvae | of larvae | of total | growth | | | | |
| | at 1^{st} | at 2^{nd} | observed | inhibitory | CK = ZH11-TC | | CK = DP0158 | |
| Line ID | instar | instar | larvae | rate (%) | P value | P ≤ 0.05 | P value | P ≤ 0.05 |
| ZH11-TC | 1 | 20 | 90 | 24.18 | | | | |
| DP0158 | 1 | 15 | 90 | 18.68 | | | | |
| DP0482 | 3 | 113 | 488 | 24.24 | 0.8628 | | 0.3166 | |

The second OAW assay was performed. As shown in Table 13, five days after larvae inoculation, 31 larvae of 449 larvae found in the OsCRK6 transgenic rice well developed to 1^{st} instar, and 88 larvae developed to 2^{nd} instar. The OAW larvae growth inhibitory rate was 31.25%. While, six of the 88 larvae in the ZH11-TC wells developed to 1^{st} instar, and 15 larvae developed to 2^{nd} instar. The larvae growth inhibitory rate of ZH11-TC seedlings was 28.72%. Four of 84 larvae in the DP0158 seedling well developed to 1^{st} instar, and 18 larvae developed to 2^{nd} instar. The larvae growth inhibitory rate of DP0158 seedlings was 29.55%. These results also demonstrate that OsCRK6 transgenic rice exhibited greater OAW larvae growth inhibitory rate than both ZH11-TC and DP0158 controls.

TABLE 13
Armyworm assay of OsCRK6 transgenic rice under laboratory screen condition at
construct level (2^{nd} experiment)

| | Number | Number | Number | Larvae | | | | |
|---|---|---|---|---|---|---|---|---|
| | of larvae | of larvae | of total | growth | | | | |
| | at 1^{st} | at 2^{nd} | observed | inhibitory | CK = ZH11-TC | | CK = DP0158 | |
| Line ID | instar | instar | larvae | rate (%) | P value | P ≤ 0.05 | P value | P ≤ 0.05 |
| ZH11-TC | 6 | 15 | 88 | 28.72 | | | | |
| DP0158 | 4 | 18 | 84 | 29.55 | | | | |
| DP0482 | 31 | 88 | 449 | 31.25 | 0.7953 | | 0.8986 | |

### Example 10

### RSB Assay of OsCRK6 Transgenic Rice Plants Under Greenhouse Conditions

RSB assay was performed to investigate whether OsCRK6 has RSB tolerance function. The eggs of RSB were obtained from the Institute of Plant Protection of Chinese Academy of Agricultural Sciences and hatched in an incubator at 27° C.

Five OsCRK6 transgenic lines which showed better ACB insect tolerance were tested, and were cultured in greenhouse. Two types of lamps are provided as light source, i.e. sodium lamp and metal halide lamp, the ratio is 1:1. Lamps provide the 16 h/8 h period of day/night, and are placed approximately 1.5 m above the seedbed. The light intensity 30 cm above the seedbed is measured as 10,000-20,000 lx in sunny day, while 6,000-10,000 lx in cloudy day, the relative humidity ranges from 30% to 90%, and the temperature ranges from 20 to 35° C. The tillered seedlings cultured with IRRI nutrient solution for 40-d were used in this assay.

### Screening Method:

96 plants of each line were tested. When cultured for 40-d, one neonate RSB larva was inoculated on the new leaf of one rice plant, and then the plants were covered by a yarn net cage to avoid the moth entering in the greenhouse. After cultured for 28-d at 30˜35° C. in greenhouse, the withered heart rate was calculated using one way ANOVA. When the P value ≤0.05, the transgenic plants will be considered as RSB tolerant.

Rice plants with withered heart are considered as plants damaged by RSB.

The withered heart rate is percentage of number of damaged plants with withered heart over the number of total plants.

### Screening Results:

Five transgenic lines were selected and tested. After fed with RSB for 28-d, the heart of 63 ZH11-TC rice plants withered. As shown in Table 14, the withered heart rate of the five transgenic rice plants were lower than that of ZH11-TC control and two transgenic lines showed significantly lower than that of DP0158 control. These results indicate that OsCRK6 transgenic rice plants had improved tolerance against RSB insect.

TABLE 14
Rice stem borer assay of OsCRK6 transgenic
rice under greenhouse screen condition at line level

| | | Number of | | | |
|---|---|---|---|---|---|
| | | plant with | Withered | | |
| | Number of | withered | heart rate | | |
| Line ID | total plant | heart | (%) | P value | P ≤ 0.05 |
| ZH11-TC | 96 | 63 | 65.63 | | |
| DP0482.06 | 96 | 55 | 57.29 | 0.1144 | |
| DP0482.34 | 96 | 52 | 54.17 | 0.0328 | Y |
| DP0482.35 | 96 | 50 | 52.08 | 0.0893 | |
| DP0482.38 | 96 | 46 | 47.92 | 0.0224 | Y |
| DP0482.39 | 96 | 51 | 53.13 | 0.0625 | |

In summary, OsCRK6 transgenic rice plants inhibited the development of ACB and OAW insect larvae, and obtained ACB and OAW insect tolerance at seedling stage; and OsCRK6 transgenic rice plants exhibited improved tolerance against RSB insect. These results showed OsCRK6 transgenic rice had significant inhibitory impact on the growth and development of ACB, OAW and RSB insects, indicating that OsCRK6 plays insecticidal activities in the potential broad spectrum.

### Example 11

### ACB Assay of OsMFS5 Transgenic Rice Plants Under Laboratory Conditions

In order to investigate whether OsMFS5 transgenic rice can recapitulate the insect tolerance trait of AH29691 line, the OsMFS5 transgenic rice was tested against ACB insect. The method is described in Example 8.

### ACB Screening Results:

### 1) Results of First Validation Experiment

After ACB neonate larvae inoculating seedlings for 5 days in the assays, the seedlings of ZH11-TC and DP0158 were significantly damaged by ACB insects, while the OsMFS5 transgenic seedlings were less damaged, and the insects fed with the OsMFS5 transgenic seedlings was smaller than that fed with ZH11-TC and DP0158 controls.

Twelve OsMFS5 transgenic lines were placed on one 32-well plate with 6 repeats. A total of 551 ACB neonate larvae were found in OsMFS5 transgenic seedlings wells, wherein 17 larvae developed to 1^{st} instar and 252 larvae developed to 2^{nd} instar, the average larvae growth inhibitory rate was 50.35%; while 117 larvae were found in ZH11-TC seedling wells, three larvae developed to 1^{st} instar and 41 larvae developed to 2^{nd} instar; and four larvae of all observed 98 larvae inoculated on the DP0158 seedling developed to 1^{st} instar, and 44 larvae developed to 2^{nd} instar, the other 50 larvae normally developed to 3^{rd} instar. The average larvae growth inhibitory rates of ZH11-TC seedlings and DP0158 seedling were 39.17% and 50.98%, respectively. The average larvae growth inhibitory rate of OsMFS5 transgenic rice was significantly greater than that of ZH11-TC control (P value=0.0080). These results demonstrate that over-expression of OsMFS5 increased ACB insect tolerances of transgenic rice at construct level.

Further analysis at transgenic line level is displayed in Table 15. The larvae growth inhibitory rates of two lines were more than 70%, significantly greater than that of ZH11-TC and DP0158 seedlings. Four lines exhibited significantly greater larvae growth inhibitory rates than ZH11-TC control and slightly greater larvae growth inhibitory rates than DP0158 seedlings. These results consistently demonstrate that OsMFS5 transgenic rice showed inhibitory impact on ACB larval growth and OsMFS5 plays a role in increasing ACB insect tolerance of transgenic rice seedlings at construct and line levels.

TABLE 15
Asian corn borer assay of OsMFS5 transgenic rice under laboratory screening
condition at line level (1^{st} experiment)

| | | | Number of | | | | | |
|---|---|---|---|---|---|---|---|---|
| | Number of | Number of | total | Larvae growth | CK = ZH11-TC | | CK = DP0158 | |
| | larvae at | larvae at | observed | Inhibitory rate | P | | P | |
| Line ID | 1^{st} instar | 2^{nd} instar | larvae | (%) | value | P ≤ 0.05 | value | P ≤ 0.05 |
| ZH11-TC | 3 | 41 | 117 | 39.17 | | | | |
| DP0158 | 4 | 44 | 98 | 50.98 | | | | |
| DP1191.01 | 0 | 17 | 37 | 45.95 | 0.4924 | | 0.5513 | |
| DP1191.02 | 3 | 22 | 37 | 70.00 | 0.0009 | Y | 0.0320 | Y |
| DP1191.03 | 3 | 20 | 40 | 60.47 | 0.0239 | Y | 0.3464 | |
| DP1191.05 | 1 | 21 | 53 | 42.59 | 0.6024 | | 0.3463 | |
| DP1191.06 | 3 | 20 | 28 | 83.87 | 0.0002 | Y | 0.0034 | Y |
| DP1191.07 | 2 | 26 | 51 | 56.60 | 0.0402 | Y | 0.5527 | |
| DP1191.08 | 1 | 22 | 53 | 44.44 | 0.4670 | | 0.4541 | |
| DP1191.10 | 1 | 14 | 55 | 28.57 | 0.1411 | | 0.0057 | |
| DP1191.11 | 0 | 16 | 51 | 31.37 | 0.4313 | | 0.0345 | |
| DP1191.13 | 2 | 24 | 47 | 57.14 | 0.0293 | Y | 0.4447 | |
| DP1191.14 | 1 | 23 | 51 | 48.08 | 0.2026 | | 0.8464 | |
| DP1191.15 | 0 | 27 | 48 | 56.25 | 0.0306 | Y | 0.4485 | |

### 2) Results of Second Validation Experiment

The same twelve OsMFS5 transgenic lines were placed on one 32-well plate with 6 repeats. A total of 691 ACB neonate larvae were found in OsMFS5 transgenic seedlings wells, wherein one larva developed to 1^{st} instar and 308 larvae developed to 2^{nd} instar, the average larvae growth inhibitory rate was 44.80%; while 127 larvae were found in ZH11-TC seedling wells and 43 larvae developed to 2^{nd} instar. The average larvae growth inhibitory rate of ZH11-TC seedlings was 33.86%. The average larvae growth inhibitory rate of OsMFS5 transgenic rice was significantly greater than that of ZH11-TC control (P value=0.0147). These results demonstrate that over-expression of OsMFS5 increased ACB insect tolerances of transgenic rice seedlings at construct level.

Further analysis at transgenic line level is displayed in Table 16. Ten lines had greater larvae growth inhibitory rates than that of ZH11-TC control; and three lines had significantly greater larvae growth inhibitory rates than that of ZH11-TC controls. Two lines (DP1191.03 and DP1191.06) showed the highest larvae growth inhibitory rates in two experiments. The results in the third experiment also exhibited the same trend. These results demonstrate that OsMFS5 transgenic rice showed inhibitory impact on ACB larval growth and OsMFS5 plays a role in increasing ACB insect tolerance of transgenic rice seedlings at construct and line levels.

TABLE 16
Asian corn borer assay of OsMFS5 transgenic rice under laboratory screening
condition at line level (2^{nd} experiment)

| | Number | | Number | | | |
|---|---|---|---|---|---|---|
| | of larvae | Number of | of total | Larvae growth | | |
| | at 1^{st} | larvae at | observed | inhibitory | CK = ZH11-TC | |
| Line ID | instar | 2^{nd} instar | larvae | rate (%) | P value | P ≤ 0.05 |
| ZH11-TC | 0 | 43 | 127 | 33.86 | | |
| DP1191.01 | 0 | 24 | 56 | 42.86 | 0.2492 | |
| DP1191.02 | 0 | 27 | 57 | 47.37 | 0.0869 | |
| DP1191.03 | 0 | 34 | 52 | 65.38 | 0.0003 | Y |
| DP1191.05 | 0 | 32 | 61 | 52.46 | 0.0182 | Y |
| DP1191.06 | 1 | 28 | 39 | 75.00 | 0.0000 | Y |
| DP1191.07 | 0 | 29 | 60 | 48.33 | 0.0631 | |
| DP1191.08 | 0 | 24 | 67 | 35.82 | 0.7847 | |
| DP1191.10 | 0 | 19 | 56 | 33.93 | 0.9918 | |
| DP1191.11 | 0 | 27 | 62 | 43.55 | 0.2005 | |
| DP1191.13 | 0 | 26 | 54 | 48.15 | 0.0761 | |
| DP1191.14 | 0 | 19 | 65 | 29.23 | 0.5195 | |
| DP1191.15 | 0 | 19 | 62 | 30.65 | 0.6609 | |

### Example 12

OAW Assay of OsMFS5 Transgenic Rice Plants under Laboratory Conditions OAW assay of OsMFS5 transgenic rice was performed as described in Example 9. The screening results as below.

Twelve OsMFS5 transgenic rice lines tested in ACB assay were tested in OAW assay. These twelve lines were placed on the one 32-well plate with four repeats. Five days after co-culture, 452 larvae were found in the OsMFS5 transgenic rice wells, wherein two larvae developed to 1^{st} instar and 169 OAW larvae developed to 2^{nd} instar, while 22 of the 85 larvae in the ZH11-TC well developed to 2^{nd} instar, and 31 of 83 larvae in the DP0158 well developed to 2^{nd} instar. The average OAW larvae growth inhibitory rates of OsMFS5 transgenic rice, ZH11-TC and DP0158 were 38.11%, 25.88% and 37.35%. The OAW larvae growth inhibitory rate of OsMFS5 transgenic rice was significantly greater than that of ZH11-TC control (P value=0.0452).

Analysis at line level was shown in Table 17. Seven lines had greater larvae growth inhibitory rates than that of both ZH11-TC and DP0158 controls. Three lines had significantly greater inhibitory rates than ZH11-TC control. These results demonstrate that OsMFS5 transgenic rice had improved OAW tolerance than ZH11-TC control at seedling stage.

TABLE 17
Armyworm assay of OsMFS5 transgenic rice under laboratory screen condition at
line level (1^{st} experiment)

| | Number | | | | | | | |
|---|---|---|---|---|---|---|---|---|
| | of | Number | Number | Larvae | | | | |
| | larvae | of larvae | of total | growth | | | | |
| | at 1^{st} | at 2^{nd} | observed | inhibitory | CK = ZH11-TC | | CK = DP0158 | |
| Line ID | instar | instar | larvae | rate (%) | P value | P ≤ 0.05 | P value | P ≤ 0.05 |
| ZH11-TC | 0 | 22 | 85 | 25.88 | | | | |
| DP0158 | 0 | 31 | 83 | 37.35 | | | | |
| DP1191.01 | 0 | 16 | 39 | 41.03 | 0.0965 | | 0.7284 | |
| DP1191.02 | 0 | 9 | 34 | 26.47 | 0.9531 | | 0.2473 | |
| DP1191.03 | 0 | 13 | 35 | 37.14 | 0.2335 | | 0.9282 | |
| DP1191.05 | 0 | 17 | 37 | 45.95 | 0.0380 | Y | 0.4210 | |
| DP1191.06 | 0 | 8 | 33 | 24.24 | 0.8483 | | 0.1726 | |
| DP1191.07 | 0 | 15 | 41 | 36.59 | 0.2126 | | 0.9200 | |
| DP1191.08 | 0 | 17 | 42 | 40.48 | 0.0935 | | 0.7382 | |
| DP1191.10 | 0 | 10 | 42 | 23.81 | 0.8152 | | 0.1301 | |
| DP1191.11 | 0 | 20 | 39 | 51.28 | 0.0085 | Y | 0.1623 | |
| DP1191.13 | 0 | 16 | 42 | 38.10 | 0.1654 | | 0.9755 | |
| DP1191.14 | 2 | 13 | 32 | 50.00 | 0.0157 | Y | 0.2247 | |
| DP1191.15 | 0 | 15 | 36 | 41.67 | 0.0910 | | 0.6807 | |

The transgenic lines were tested again in another two experiments. In the second experiment, five days later after inoculation of OAW neonate larvae, 336 larvae were found in the OsMFS5 transgenic rice well, 11 larvae grew to 1^{st} instar and 182 larvae grew to 2^{nd} instar. The larvae growth inhibitory rate was 58.79%. Whereas, one larva of 54 in the ZH11-TC seedling wells grew to 1^{st} instar, and 29 larvae grew to 2^{nd} instar, and 23 larvae of the 62 larvae in DP0158 seedling wells grew to 2^{nd} instar. The OAW larvae growth inhibitory rate of OsMFS5 transgenic rice was slightly greater than that of ZH11-TC (56.36%) and significantly greater than DP0158 (37.10%, P value=0.0032). These results indicate that OsMFS5 transgenic rice exhibited OAW larvae tolerance at construct level.

Analysis at line level shows that four lines had the larvae growth inhibitory rates more than 60%, which were significantly greater than that of DP0158 control. The third experiment showed the similar trend. These results further confirm that over-expression OsMFS5 enhanced tolerance against OAW insect in transgenic rice plants, and OsMFS5 plays a role in increasing OAW insect tolerance.

TABLE 18
OAW assay of OsMFS5 transgenic rice plants under laboratory screen condition
at line level (2^{nd} experiment)

| | Number | Number | Number of | Larvae | | | | |
|---|---|---|---|---|---|---|---|---|
| | of larva | of larvae | total | growth | | | | |
| | at 1^{st} | at 2^{nd} | observed | inhibitory | CK = ZH11-TC | | CK = DP0158 | |
| Event ID | instar | instar | larvae | rate (%) | P value | P ≤ 0.05 | P value | P ≤ 0.05 |
| ZH11-TC | 1 | 29 | 54 | 56.36 | | | | |
| DP0158 | 0 | 23 | 62 | 37.10 | | | | |
| DP1191.01 | 0 | 24 | 33 | 72.73 | 0.1384 | | 0.0029 | Y |
| DP1191.02 | 4 | 12 | 29 | 60.61 | 0.786 | | 0.0461 | Y |
| DP1191.03 | 1 | 12 | 23 | 58.33 | 0.8765 | | 0.0842 | |
| DP1191.05 | 1 | 14 | 29 | 53.33 | 0.8512 | | 0.1257 | |
| DP1191.06 | 1 | 22 | 28 | 82.76 | 0.0283 | Y | 0.0007 | Y |
| DP1191.07 | 0 | 11 | 19 | 57.89 | 0.968 | | 0.1356 | |
| DP1191.08 | 1 | 15 | 27 | 60.71 | 0.6991 | | 0.0442 | Y |
| DP1191.10 | 0 | 14 | 32 | 43.75 | 0.2713 | | 0.5105 | |
| DP1191.11 | 1 | 17 | 32 | 57.58 | 0.9231 | | 0.0651 | |
| DP1191.13 | 0 | 14 | 28 | 50.00 | 0.5885 | | 0.2484 | |
| DP1191.14 | 2 | 10 | 25 | 51.85 | 0.722 | | 0.1886 | |
| DP1191.15 | 0 | 17 | 31 | 54.84 | 0.9092 | | 0.1055 | |

### Example 13

### RSB Assay of OsMFS5 Transgenic Rice Plants Under Greenhouse Conditions

RSB assay of OsMFS5 transgenic rice was performed as described in Example 10. The screening results as below.

Five lines shown better ACB and OAW tolerance were tested. After fed RSB for 28-d, 66 of the 98 DP0158 rice plants had withered heart, and 34 of the 96 DP1191.02 showed withered heart. The withered heart rates of all the five transgenic rice were less than that of DP0158 control (Table 19). Twelve days later, the mortality rates of the rice plants were counted. As shown in Table 20, the five transgenic lines showed less mortality rates than DP0158 control, and three lines showed significantly less mortality rates. These results consistently demonstrated that OsMFS5 transgenic rice obtained improved RSB tolerance.

TABLE 19
Rice stem borer assay of OsMFS5 transgenic rice
plants under greenhouse screen condition (Withered rate)

| | | Number of | | | |
|---|---|---|---|---|---|
| | Number of | plants with | Withered | | |
| | total | withered | heart rate | | |
| Lines ID | plants | heart | (%) | P value | P ≤ 0.05 |
| DP0158 | 96 | 66 | 68.75 | | |
| DP1191.02 | 96 | 34 | 35.42 | 0.0012 | Y |
| DP1191.03 | 96 | 54 | 56.25 | 0.3209 | |
| DP1191.06 | 96 | 51 | 53.13 | 0.1467 | |
| DP1191.13 | 96 | 48 | 50 | 0.0731 | |
| DP1191.15 | 96 | 58 | 60.42 | 0.4252 | |

TABLE 20
Rice stem borer assay of OsMFS5 transgenic rice plants
under greenhouse screen condition (Mortality rate)

| | Number of | | | | |
|---|---|---|---|---|---|
| | total | Number of | Mortality | | |
| Lines ID | plants | dead plant | rate (%) | P value | P ≤ 0.05 |
| DP0158 | 48 | 18 | 37.5 | | |
| DP1191.02 | 48 | 5 | 10.42 | 0.0113 | Y |
| DP1191.03 | 48 | 5 | 10.42 | 0.0113 | Y |
| DP1191.06 | 48 | 16 | 33.33 | 0.6480 | |
| DP1191.13 | 48 | 7 | 14.58 | 0.0533 | Y |
| DP1191.15 | 48 | 15 | 31.25 | 0.6946 | |

OsMFS5 transgenic rice plants showed inhibitory impact on ACB and OAW larval growth and OsMFS5 plays a role in increasing ACB and OAW insect tolerance of transgenic rice seedlings; and OsMFS5 transgenic rice plants exhibited improved tolerance against RSB insect. These results showed OsMFS5 transgenic rice had significant inhibitory impact on the growth and development of ACB, OAW and RSB insects, indicating that OsMFS5 plays insecticidal activities in the potential broad spectrum.

What is claimed is:

1. An isolated polynucleotide, comprising: (a) a polynucleotide with nucleotide sequence of at least 85% sequence identity to SEQ ID NO: 4 and 12; (b) a polynucleotide with nucleotide sequence of at least 85% sequence identity to SEQ ID NO: 5 and 13; (c) a polynucleotide encoding a polypeptide with amino acid sequence of at least 90% sequence identity to SEQ ID NO: 6 and 14; or (d) the full complement of the nucleotide sequence of (a), (b) or (c), wherein over-expression of the polynucleotide in a plant increases tolerance to an insect pest.

2. The isolated polynucleotide of claim 1, the nucleotide sequence comprises SEQ ID NO: 4, SEQ ID NO: 5, SEQ ID NO: 12 or SEQ ID NO: 13.

3. The isolated polynucleotide of claim 1, wherein the isolated polynucleotide encoded polypeptide comprises the amino acid sequence of SEQ ID NO: 6 or SEQ ID NO: 14.

4. The isolated polynucleotide of any one of claims 1 to 3, wherein the polynucleotide is from Oryza sativa, Oryza australiensis, Oryza barthii, Oryza glaberrima, Oryza latifolia, Oryza longistaminata, Oryza meridionalis, Oryza officinalis, Oryza punctata, Oryza rufipogon, Oryza nivara, Arabidopsis thaliana, Cicer arietinum, Solanum tuberosum, Brassica oleracea, Zea mays, Glycine max, Glycine tabacina, Glycine soja or Glycine tomentella.

5. The isolated polynucleotide of claim 1, wherein the insect pest is a Lepidopteran.

6. The isolated polynucleotide of claim 5, wherein the insect pest is Asian Corn Borer (Ostrinia furnacalis), Rice Stem Borer (Chilo suppressalis), or Oriental Armyworm (Mythimna separata).

7. A recombinant DNA construct, comprising the isolated polynucleotide of any one of claims 1 to 4 operably linked to at least one heterologous regulatory sequence.

8. A recombinant DNA construct, comprising an isolated polynucleotide encoding a CRK6 or MFS5 polypeptide operably linked to at least one heterologous regulatory sequence.

9. A transgenic plant, plant cell or seed, comprising a recombinant DNA construct, wherein the recombinant DNA construct comprises the polynucleotide of any one of claims 1 to 4 operably linked to at least one heterologous regulatory sequence.

10. A transgenic plant or plant cell, comprising in its genome a recombinant DNA construct comprising polynucleotide of any one of claims 1 to 4 operably linked to at least one heterologous regulatory element, wherein said plant exhibits increased tolerance to an insect pest when compared to a control plant.

11. The transgenic plant or plant cell of claim 10, wherein the insect pest is a Lepidopteran.

12. The transgenic plant or plant cell of claim 11, wherein the insect pest is Asian Corn Borer (Ostrinia furnacalis), Rice Stem Borer (Chilo suppressalis), or Oriental Armyworm (Mythimna separata).

13. The plant of claims 9 to 12, wherein said plant is selected from the group consisting of rice, maize, soybean, sunflower, sorghum, canola, wheat, alfalfa, cotton, barley, millet, sugar cane and switchgrass.

14. A method of increasing tolerance in a plant to an insect pest, comprising overexpressing at least one polynucleotide encoding a CRK6 or MFS5 polypeptide.

15. The method of claim 14, wherein the polynucleotide comprises: (a) a polynucleotide with a nucleotide sequence of at least 85% sequence identity to SEQ ID NO: 4 or 12; (b) a polynucleotide with a nucleotide sequence of at least 85% sequence identity to SEQ ID NO: 5 or 13; and (c) a polynucleotide encoding a polypeptide with amino acid sequence of at least 90% sequence identity to SEQ ID NO: 6 or 14.

16. The method of claim 14 or 15, wherein the plant comprises the DNA construct of claim 8.

17. A method of increasing tolerance in a plant to an insect pest, comprising:
(a) introducing into a regenerable plant cell a recombinant DNA construct comprising a polynucleotide operably linked to at least one regulatory sequence, wherein the polynucleotide encodes a polypeptide having an amino acid sequence of at least 80% sequence identity compared to SEQ ID NO: 6 or 14;
(b) regenerating a transgenic plant from the regenerable plant cell after step (a), wherein the transgenic plant comprises in its genome the recombinant DNA construct; and
(c) obtaining a progeny plant derived from the transgenic plant of step (b), wherein said progeny plant comprises in its genome the recombinant DNA construct and exhibits increased tolerance to an insect pest when compared to a control plant not comprising the recombinant DNA construct.

18. The method of claim 17, wherein the insect pest is a Lepidopteran.

19. The method of claim 18, wherein the insect pest is Asian Corn Borer (Ostrinia furnacalis), Rice Stem Borer (Chilo suppressalis), or Oriental Armyworm (Mythimna separata).

20. A method of evaluating tolerance in a plant to an insect pest, comprising:
(a) introducing into a regenerable plant cell a recombinant DNA construct comprising a polynucleotide operably linked to at least one regulatory sequence, wherein the polynucleotide encodes a polypeptide having an amino acid sequence of at least 80% sequence identity when compared to SEQ ID NO: 6 or 14;
(b) regenerating a transgenic plant from the regenerable plant cell after step (a), wherein the transgenic plant comprises in its genome the recombinant DNA construct;
(c) obtaining a progeny plant derived from the transgenic plant, wherein the progeny plant comprises in its genome the recombinant DNA construct; and
(d) evaluating the progeny plant for tolerance to an insect pest compared to a control plant not comprising the recombinant DNA construct.

21. The method of claim 20, wherein the insect pest is a Lepidopteran.

22. The method of claim 21, wherein the insect pest is Asian Corn Borer (Ostrinia furnacalis), Rice Stem Borer (Chilo suppressalis), or Oriental Armyworm (Mythimna separata).
